(19) United States
(12) Patent Application Publication
(10) Pub. No.: US 2020/0237819 A1
(43) Pub. Date: Jul. 30, 2020

(54) T CELL COMPOSITIONS FOR IMMUNOTHERAPY

(71) Applicant: GENEIUS BIOTECHNOLOGY, INC., Natick, MA (US)

(72) Inventors: Alfred E. SLANETZ, Cohasset, MA (US); Terry Y. NAKAGAWA, Evanston, IL (US); Marissa A. HERRMAN, Boston, MA (US)

(73) Assignee: Geneius Biotechnology, Inc., Natick, MA (US)

(21) Appl. No.: 16/312,023

(22) PCT Filed: Jun. 28, 2017
(86) PCT No.: PCT/US2017/039846

§ 371 Date: Dec. 20, 2018

Related U.S. Application Data
(60) Provisional application No. 62/355,533, filed on Jun. 28, 2016.
(60) Provisional application No. 62/355,506, filed on Jun. 28, 2016.
(60) Provisional application No. 62/355,458, filed on Jun. 28, 2016.

(51) Int. Cl. A61K 35/17; C12N 5/0783; A61P 35/00

(52) U.S. Cl. CPC A61K 35/17; C12N 5/0636; A61P 35/00; C12N 2501/2321; C12N 2501/2302; C12N 2501/2307; C12N 2501/2315

(57) ABSTRACT

The invention relates to compositions comprising a heterogeneous population of T cells with reactivity to selected antigens that are useful for adoptive immunotherapy and methods for making the T cell compositions.

### FIELD OF THE INVENTION

The invention relates to compositions comprising a heterogeneous population of T cells with reactivity to selected antigens that are useful for adoptive immunotherapy and methods for making the T cell compositions.

### BACKGROUND

Over a period of days after a person's immune system first sees an antigen, a population of T cells that recognize the antigen is generated, and these T cells determine the nature of the response to that antigen thereafter. Antigen recognition and specificity by a T cell is conferred by the structural characteristic of the T cell receptor (TCR) expressed on the cell surface. A single T cell has TCRs capable of binding to a single antigen presented in combination with a specific Major Histocompatibility Complex molecule, or MHC. Therefore, antigen specificity of a T cell is characterized by the presence and function of the specific TCR exhibited by the cell. While there are multiple subtypes of cells involved, generally the T cells that appear are characterized by various cell surface markers (CD4+: TH1, TH2, Treg, T follicular helper, TH17, TH22, TH9; CD8+: CTLs, etc.) and it is due to the function of these different cellular subtypes, that a cellular or humoral immune response results. In addition, certain subsets of T cells in the population are immunosuppressive (e.g., Treg, TH17, anergized T cells), and their presence can induce immune tolerance.

Adoptive transfer of ex-vivo expanded antigen-specific T cells was shown to confer immunity against CMV and EBV as early as in the 1990s. (Riddell et al. Science 1992; 257: 238) (Rooney et al. Blood 1998; 92: 1549-55). However, over the course of tumor progression, the immune response to the tumor became focused on a small number of “dominant” antigens, which were ineffective in promoting tumor regression. In past attempts of using ex vivo expanded T cells for immunotherapy, tumor associated dominant antigen-responsive T cells were inadvertently expanded, leading to inconsistencies in the outcome.

In a study by Kawakami et al, most melanoma patients exhibited cytotoxic T lymphocyte (CTL) activity against human melanocyte-specific antigen (MART-1/Melan A), but only a few against another tumor associated antigen gp100. When tumor infiltrating lymphocytes (TILs) were used for adoptive therapy, tumor regression correlated with gp100-reactive T cells and not MART-1 reactive ones. (Kawakami Y. et al., J. Immunol. 1995, 154(8): 3961-8). In another study, immunization of melanoma patients with cancer antigens increased the number of circulating CD8+ CTLs, but did not correlate with tumor regression. (Rosenberg et al., 1998, Nature Medicine 4: 321).

These inconsistencies relate to the fact that the tumor microenvironment is complex and primarily promotes tumor survival by downregulating the cytotoxic effect of T cells. Regulatory T cell (or Treg) mediated tolerogenic responses develop, and are often directed primarily against the high abundance, high avidity-exhibiting tumor infiltrating T cells, which recognize immunodominant tumor antigen. Additionally, loss of the antigen may result providing a means for the tumor to evade immunoreactivity. Thus, when T cells isolated from a tumor (i.e., TILs) are selected ex-vivo for high antigen recognition and expansion, and then reinfused in the patient, the cells are mostly directed against the dominant tumor antigen(s) resulting in only a temporal reduction of tumor burden. The tumor may become refractory to the subsequent administrations, even when multiple antigen-targeting T cell populations were used in the treatment regimen (Rosenberg et al., J. Immunother. 2003, 26(5): 385-393). Prior studies have also noted that the benefit of adoptive T cell therapy is augmented by prior lymphodepletion to counteract suppressive lymphocyte function. In earlier cases, preconditioning the host with chemotherapy increased the response to the subsequent immunotherapy (Dudley M. E. et al., Science. 2002, Oct. 25; 298(5594):850-4; Dudley M. E. et al., J. Clin. Oncol., 2005, Apr. 1; 23(10):2346-57); (U.S. Pat. No. 8,034,334).

Accordingly, there remains a need for better adoptive T cell therapies.

### SUMMARY OF THE INVENTION

In one aspect, the invention provides, a method for making a composition useful in adoptive cell therapy enriched for T cells that are reactive to one or more target antigens. In one embodiment, the invention provides a method for making a composition comprising T-cells, the method comprising the steps of:
(a) obtaining an initial cell population comprising T-cells;
(b) stimulating the T-cells by exposing the cell population to an one or more target antigens and to cytokines,
(c) culturing the cell population in media comprising cytokines;
(d) testing the cell population for antigen-specific reactivity;
(e) harvest the resulting composition comprising T cells.

In one embodiment, the initial cell population comprising T cells is peripheral blood mononuclear cells (PBMCs) from a patient's blood. In one embodiment, the initial cell population is frozen and is thawed prior to starting the method. In one embodiment, the method further comprising testing the initial population of cells for total T cells (CD3+), and amounts of CD8+ and CD4+ T cells, Monocytes, B cells, and NK cells.

In one embodiment, testing the cell population for antigen-specific reactivity comprises detection of T cell activation markers. In one embodiment detection of T cell activation markers is accomplished by one or more of flow cytometry, and measurement of antigen induced cytokine production by intracellular cytokine staining, ELISA, or ELISPOT. Markers for T cell activation measure by flow cytometry include one or more of CD45RO, CD137, CD25, CD279, CD179, CD62L, HLA-DR, CD69, CD223(LAG3), CD134(OX40), CD183(CXCR3), CD27(IL-7Ra), CD366(TIM3), CD80, CD152(CTLA-4), CD28, CD278(ICOS), CD154(CD40L). Antigen induced cytokines (TNFa, IFNg, IL-2, and CD107a) are mobilized in CTLs in response to stimulation and can also be measured along with the cytokines by flow cytometry.

In embodiments, the cytokines in steps (b) and (c) individually comprise one or more of IL-2, IL-7, IL-15, and IL-21. In another embodiment, the cytokines in steps (b) and (c) comprise IL-7 and IL-15. In embodiments, the cytokines used in steps (b) and (c) are the same. In other embodiments, the cytokines used in steps (b) and (c) are the different or overlapping groups of cytokines.

In embodiments of the invention, the above methods further comprise repeating step (b). In embodiments, the above methods further comprise polyclonal stimulation of the T cells in the cell population. In one embodiment, the polyclonal stimulation comprises exposing the cell population to tetrameric antibodies that bind CD3, CD28 and CD2 after step (c).

In embodiments of the invention, the cell population is divided into multiple subpopulations, which are each stimulated by exposure to one or more different target antigens. In further embodiments, the multiple stimulated sub-populations are combined prior to step (c). In other embodiments, the multiple stimulated sub-populations are combined prior to step (e).

In embodiments of the invention, the one or more target antigens comprises a plurality of overlapping polypeptides derived from a one or more target antigens. In embodiments of the invention, the overlapping peptides are 15-50 amino acids in length. In a preferred embodiment, the polypeptides are 15 amino acids in length.

In embodiments of the invention, the one or more target antigens comprises polypeptides derived from one or more target viral antigens. In embodiments, the one or more target antigens comprises polypeptides derived from one or more target viral antigens. In further embodiments, the target antigen is a protein expressed by one or more of cytomegalovirus, Epstein-Barr virus, hepatitis B virus, human papillomavirus, adenovirus, herpes virus, human immunodeficiency virus, influenza virus, human respiratory syncytial virus, vaccinia virus, Varicella-zoster virus, Yellow fever virus, Ebola virus, and Zika virus. In embodiments, the one or more target antigens comprise polypeptides derived from one or more of the Epstein-Barr virus antigens, LMP1, LMP2, and EBNA1. In other embodiments, the one or more target antigens comprise polypeptides derived from one or more of the cytomegalovirus antigens, pp65, Cancer/testis antigen 1 (NY-ESO-1), and Survivin.

In embodiments of the invention, the one or more target antigens comprise polypeptides derived from one or more sub-dominant antigens or one or more neoantigens.

In another embodiment, the invention provides a method for making a composition comprising T cells, the method comprising the steps of:
(a) obtaining an initial cell population comprising T-cells;
(b) sorting T cells based on expression of T cell activation markers,
(d) polyclonal stimulation of T cells,
(e) harvest the resulting composition comprising T cells.

In embodiments of the invention, the method further comprises stimulating the T-cells by exposing the cell population to one or more target antigens and to cytokines.

In one embodiment, the initial cell population comprising T cells is peripheral blood mononuclear cells (PBMCs) from a patient's blood. In one embodiment, the initial cell population is frozen and is thawed prior to starting the method. In one embodiment, the method further comprising testing the initial population of cells for total T cells (CD3+), and amounts of CD8+ and CD4+ T cells, Monocytes, B cells, and NK cells.

In embodiments, steps (b) is performed on the initial cell population (e.g., PBMCs). In other embodiments, step (b) is performed 6-11 days, and preferably about 7 days after step (b).

In embodiments, the cytokines comprise one or more of IL-2, IL-7, IL-15, and IL-21. In preferred embodiments, the cytokines comprise IL-7 and IL-15.

In embodiments of the invention, the T cell activation markers in step (b) comprises one or more of CD69, CD279(PD-1), CD223(LAG3), CD134(OX40), CD183(CXCR3), CD27(IL-7Ra), CD137(4-1BB), CD366(TIM3), CD25(IL-2Ra), CD80, CD152(CTLA-4), CD28, CD278(IOS), CD154(CD40L), and CD45RO).

In embodiments of the invention, the polyclonal stimulation comprises exposing the cell population to tetrameric antibodies that bind CD3, CD28 and CD2.

In embodiments of the invention, the one or more target antigens used in the above methods comprises a plurality of overlapping peptides derived from a target antigen. In embodiments of the invention, the overlapping peptides are 15-50 amino acids in length. In a preferred embodiment, the polypeptides are 15 amino acids in length.

In embodiments of the invention, the one or more target antigens used in the above methods comprises polypeptides derived from one or more target viral antigens. In embodiments, the one or more target antigens comprise polypeptides derived from one or more target viral antigens from one or more of cytomegalovirus, Epstein-Barr virus, hepatitis B virus, human papillomavirus, adenovirus, herpes virus, human immunodeficiency virus, influenza virus, human respiratory syncytial virus, vaccinia virus, Varicella-zoster virus, Yellow fever virus, Ebola virus, and Zika virus. In embodiments, the one or more target antigens comprise polypeptides derived from one or more of the Epstein-Barr virus antigens, LMP1, LMP2, and EBNA1. In other embodiments, the one or more target antigens comprise polypeptides derived from one or more of the cytomegalovirus antigen, pp65, Cancer/testis antigen 1 (NY-ESO-1), and Survivin.

In embodiments of the invention, the one or more target antigens comprise polypeptides derived from one or more sub-dominant antigens or one or more neoantigens. In embodiments, polypeptides derived from neoantigens range from 15-50 amino acids in length. Preferred lengths include 15-25 amino acids.

In embodiments of the invention, the above methods provide a T cell composition useful for adoptive T-cell therapy. In embodiments of the invention, the above methods provide a T cell composition comprising greater than 70% CD3+ T cells with predominantly CD8+ versus CD4+ T cells. In further embodiments, the methods provide a T cell composition wherein greater than about 1% of the total CD3+ cells have reactivity toward the target antigen or antigens by measuring, e.g., intracellular cytokine response (mainly TNFα and IFNγ) to antigen as well as CD107a mobilization. In embodiments, the methods provide a T cell composition wherein greater than about 5% of the total CD3+ cells have reactivity toward the target antigen or antigens. In embodiments, the T cell composition resulting from the above methods comprises T cells having elevated surface expression of CD62L, CCR7 or CXCR3 and decreased surface expression of one or more activation/exhaustion markers LAG3, CD244(2B4), CD160, TIM-3, CTLA-4.

In one aspect, the invention provides a method for treating non-Hodgkin's lymphoma, gastric cancer, or nasopharyngeal carcinoma by administering to a patient in need thereof a T cell composition enriched for T cells reactive to one or more EBV antigens. In embodiments of the invention, the T cell composition is made by the methods of the invention where in the T cells are stimulated by exposing the cell population to polypeptides derived from one or more of the Epstein-Barr virus antigens, LMP1, LMP2, and EBNA1.

In one aspect, the invention provides a method for treating glioblastoma by administering to a patient in need thereof a T cell composition enriched for T cells reactive to one or more of the cytomegalovirus antigen, pp65, Cancer/testis antigen 1 (NY-ESO-1), and Survivin. In embodiments of the invention the T cell composition is made by the methods of the invention wherein the T cells are stimulated by exposing the cell population to polypeptides derived from one or more of pp65, Cancer/testis antigen 1 (NY-ESO-1), and Survivin.

In one aspect, the invention provides a composition comprising T cells for immunotherapy wherein the composition comprises greater than about 500,000 (and preferably greater than about 750,00, and more preferably greater than about a billion) CD3+ cells, the live cells comprise greater than 70% CD3+ T cells; the T cells are predominantly CD8+ versus CD4+ T cells and are predominantly effector memory T cells. In preferred embodiments, the T cells in the composition display minimal exhaustion markers, high expression levels of lymphocyte homing and trafficking markers, and high antigen reactivity.

### BRIEF DESCRIPTION OF THE FIGS

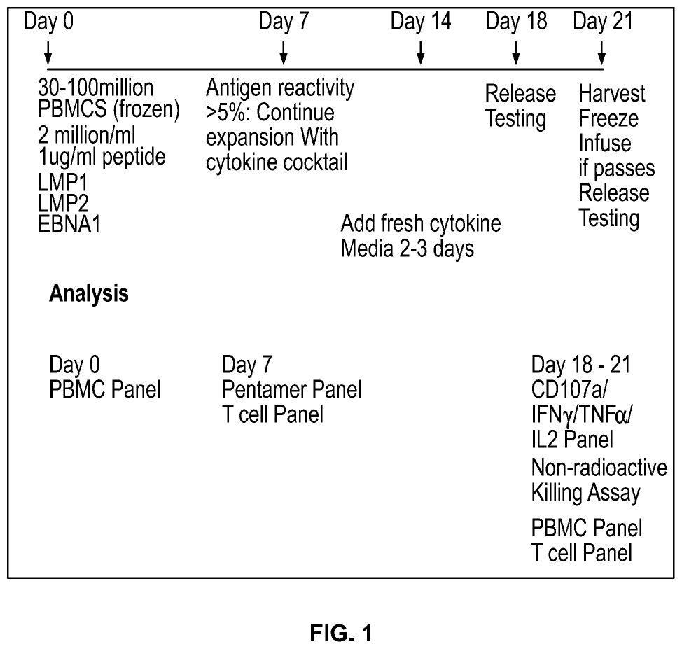

FIG. 1. A general schematic providing the steps and timing for an embodiment for generating heterogeneous T cells by stimulating and expansion ex vivo.

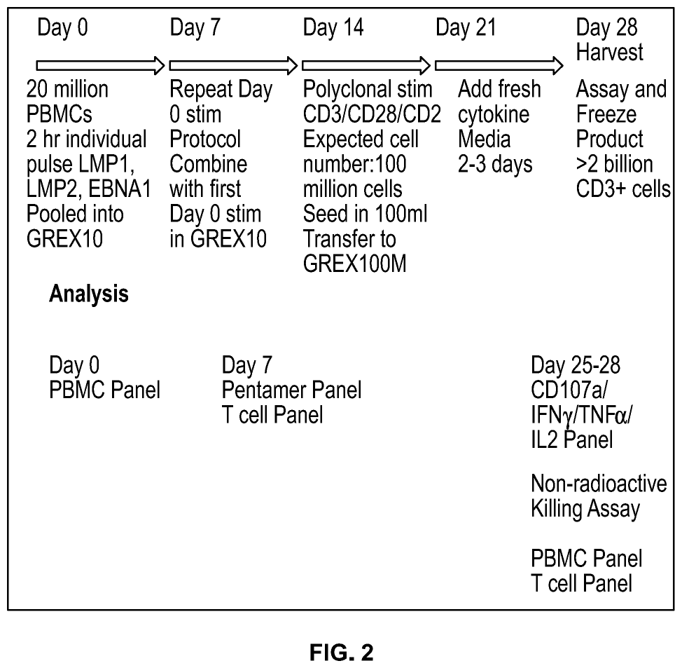

FIG. 2. A general schematic providing the steps and timing for another embodiment for generating heterogeneous T cells by stimulating and expansion ex vivo.

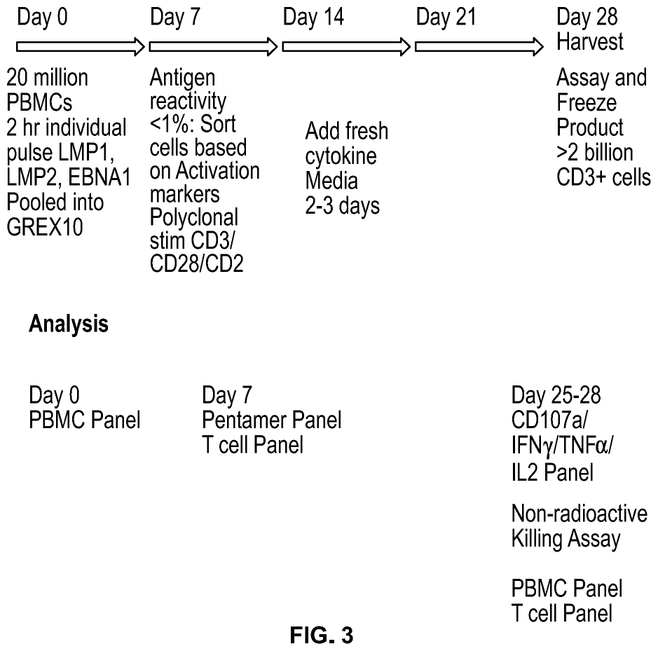

FIG. 3. A schematic providing an example of the steps and timing for one embodiment of the method for isolating and expanding heterogeneous T cells ex vivo.

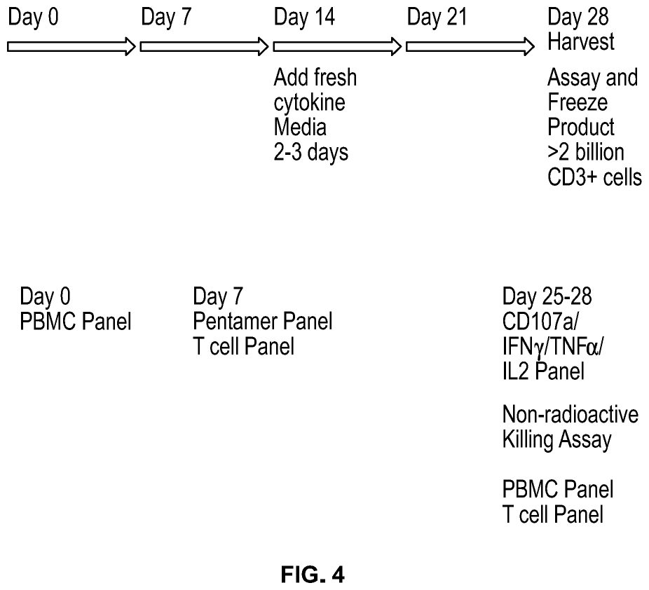

FIG. 4. A schematic providing an example of the steps and timing for another embodiment of the method for isolating and expanding heterogeneous T cells ex vivo.

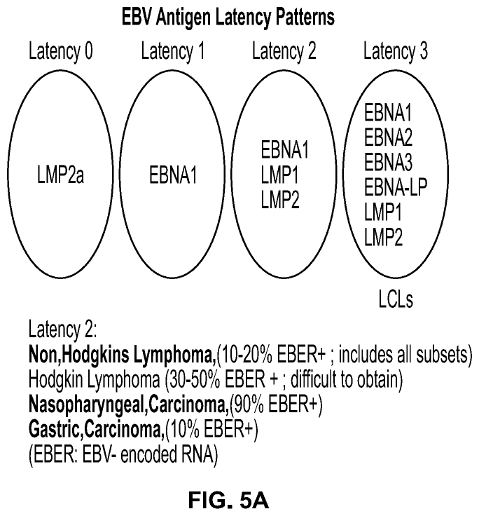

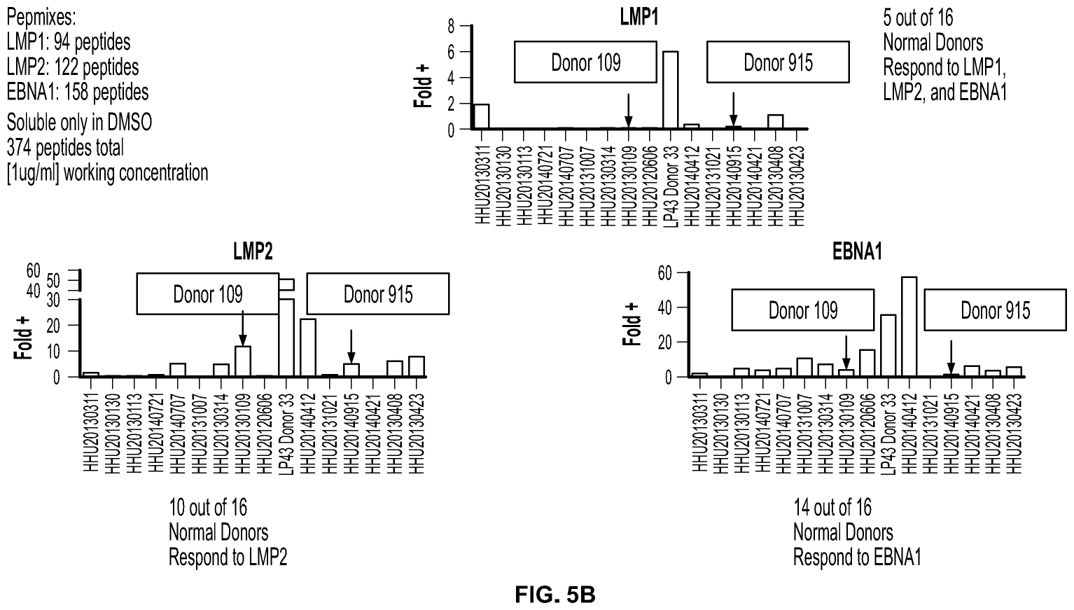

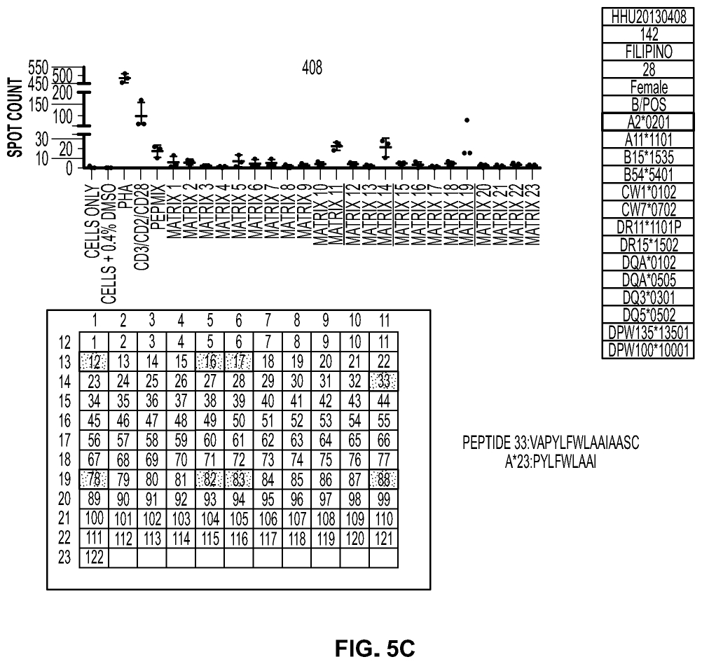

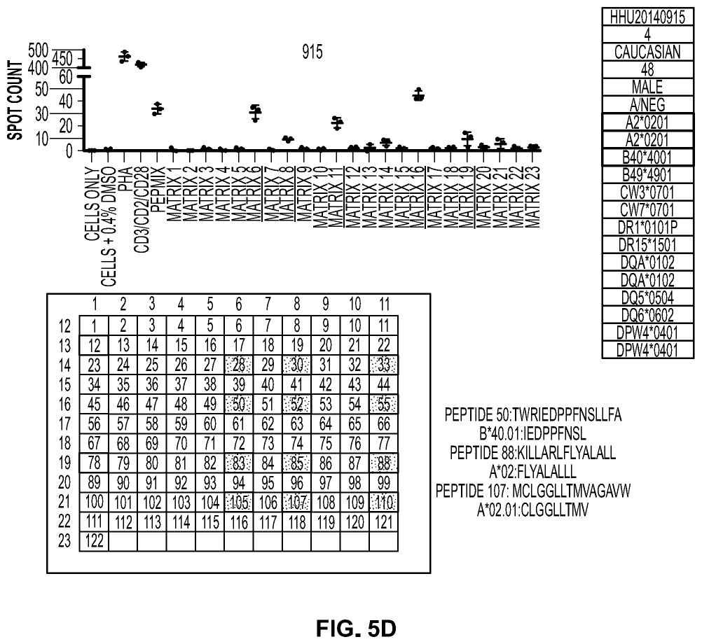

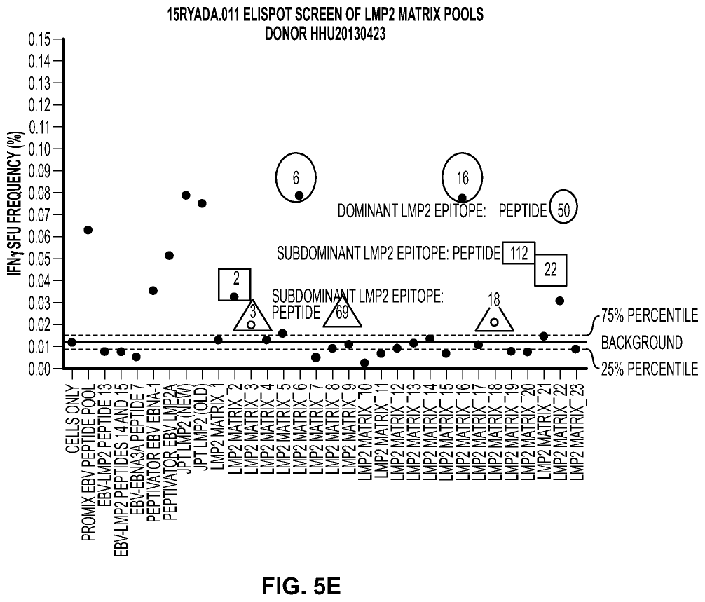

FIG. 5a. Diagram of the EBV viral antigens that are selectively expressed during Viral Latency 0, 1, 2, and 3. EBV Antigen Latency 2 is characterized by expression of EBNA1, LMP1, and LMP2 proteins and is identified in several EBER+ cancers.

FIG. 5b. LMP1, LMP2, EBNA1 polypeptide mixes (“pepmixes”) were used to screen T cell reactivity of 16 normal healthy donor PBMCs.

FIG. 5c. Normal donor 408 was HLA genotyped and the LMP2 reactive epitope identified by LMP2 matrix pool ELISPOT analysis to determine the specific CD8+ T cell ligand that is recognized.

FIG. 5d. Normal donor 915 was HLA genotyped and the multiple CD8+ HLA/LMP2 peptide T cell ligands were identified by Matrix pool screening. Donor 915 CD8 T cells recognize 3 different LMP2 peptides on two different HLA alleles.

FIG. 5e. Matrix pool screening performed with Normal donor 109 PBMCs demonstrates a high, medium, and low T cell frequency response.

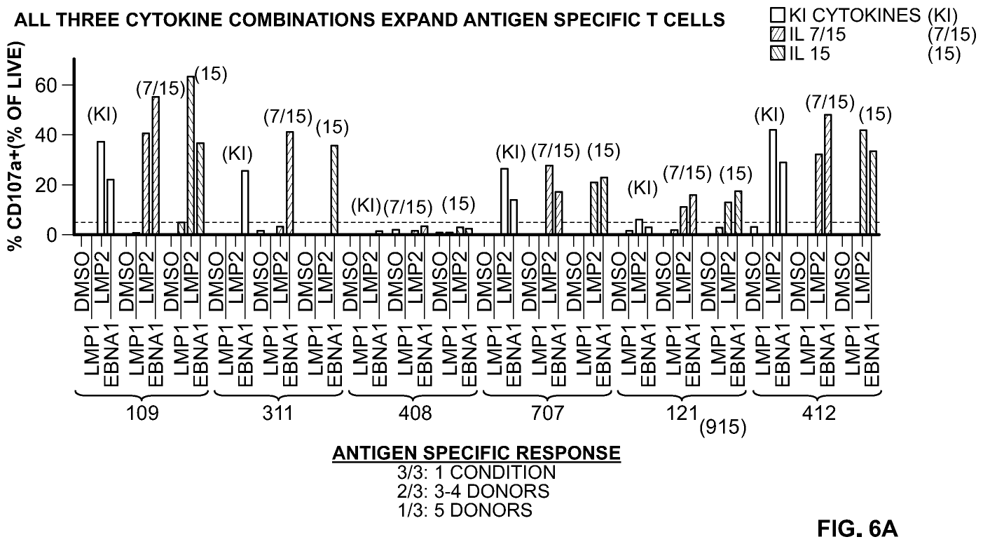

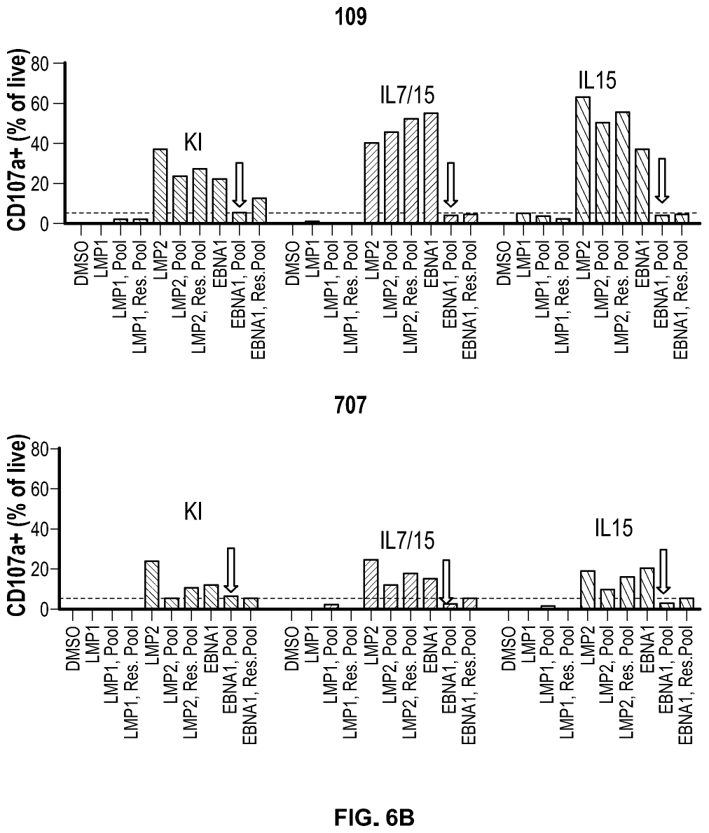

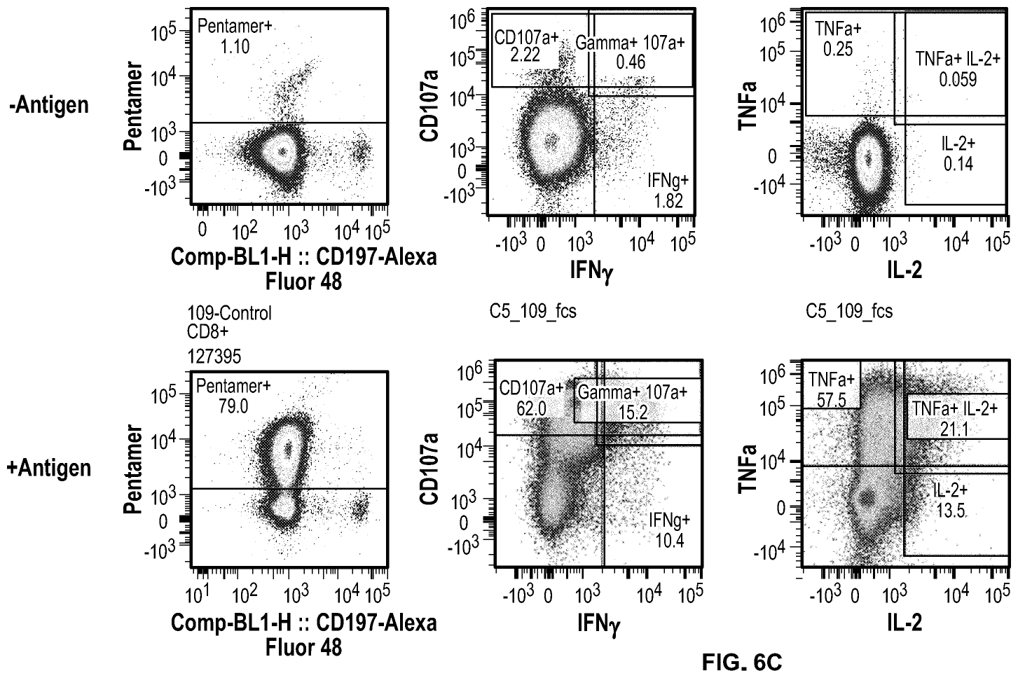

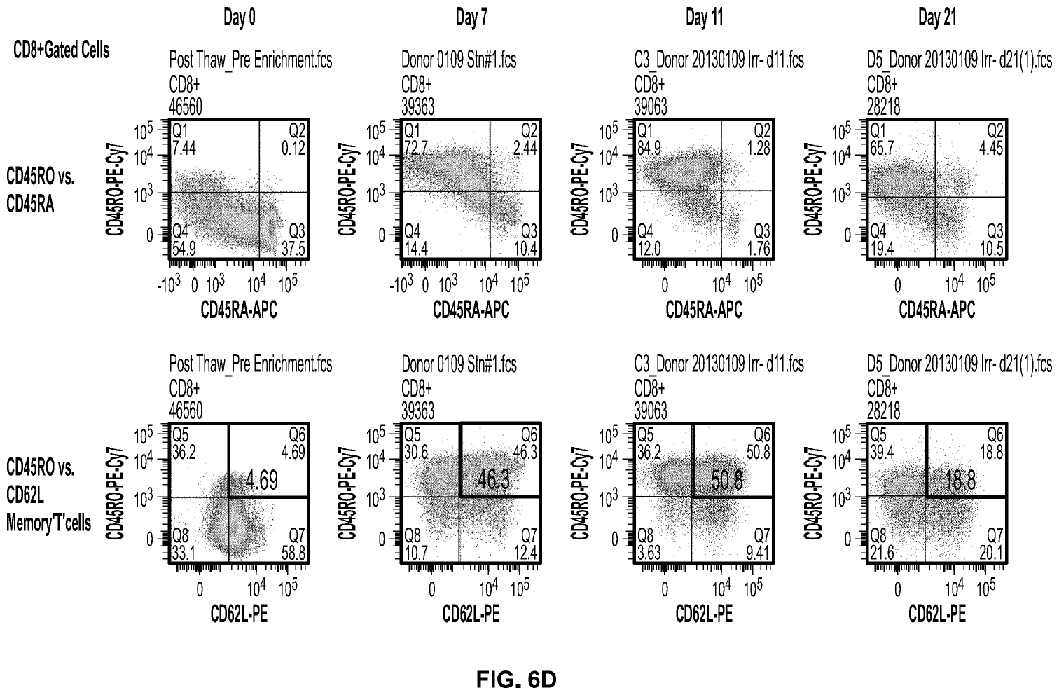

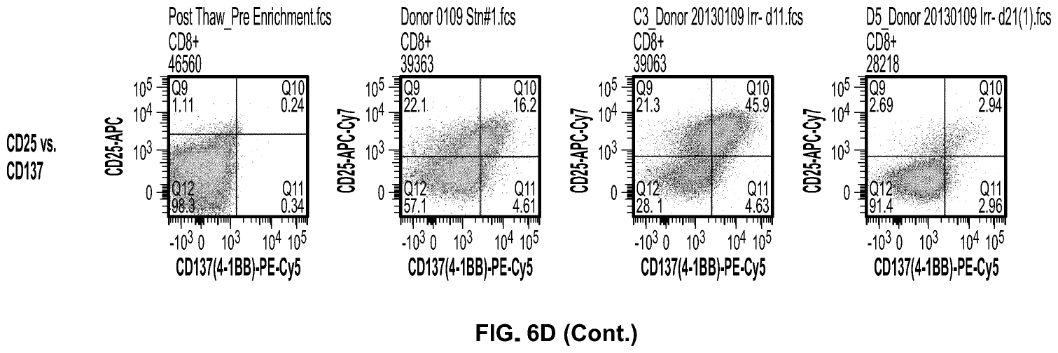

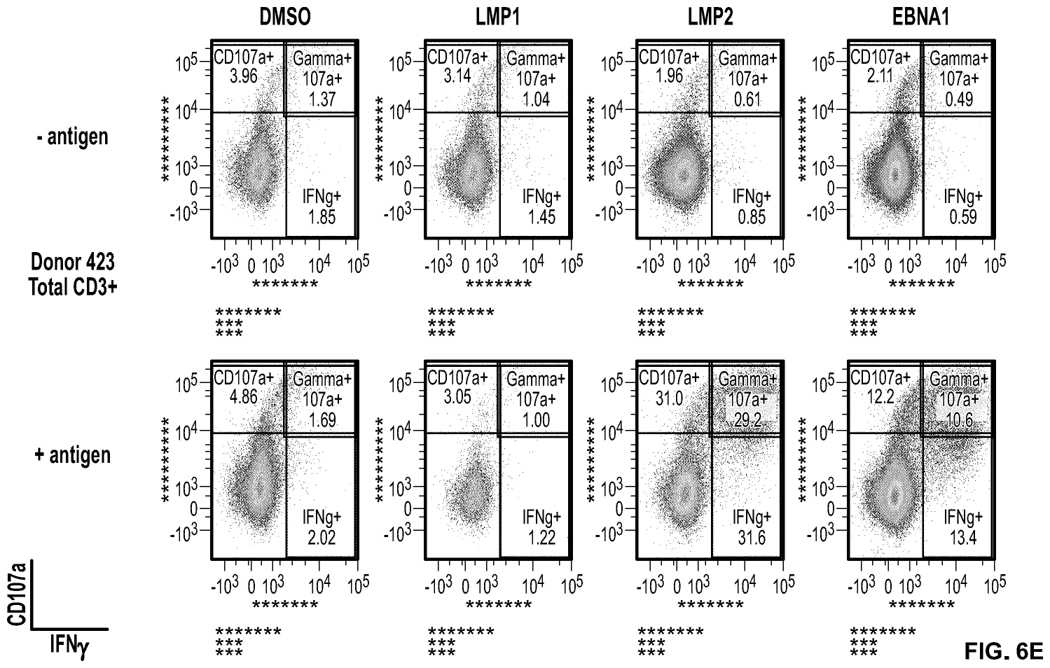

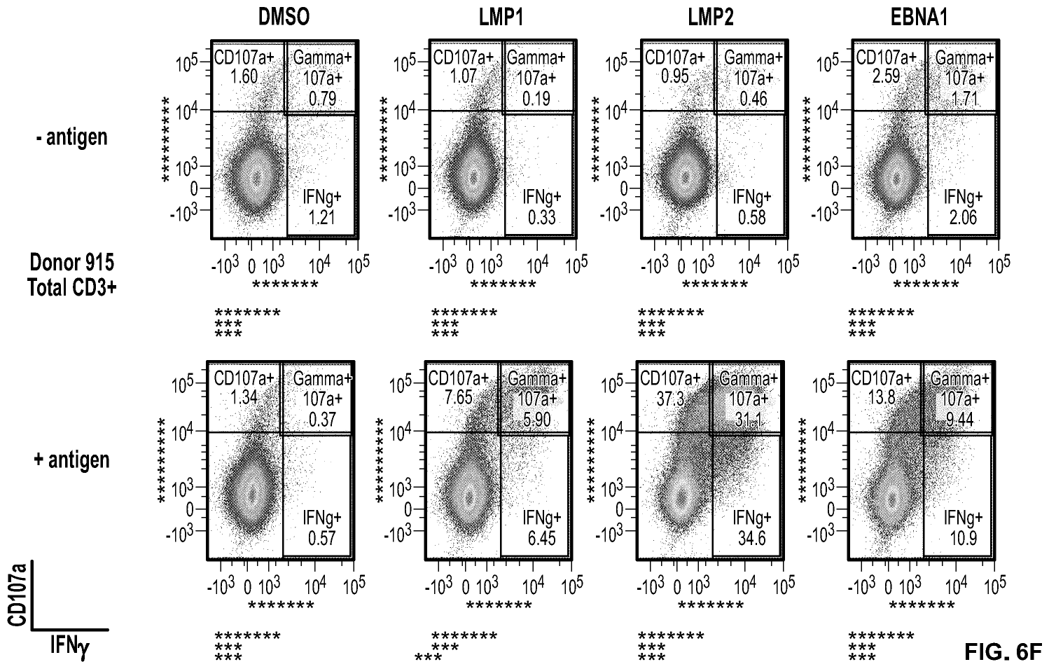

FIG. 6a. Small scale expansion with 3 cytokine conditions (KI: 1000 IU/ml IL-2, 10 ng/ml IL-15/IL-21; 10 ng/ml IL7/15; 10 ng/ml IL15 alone) evaluating 6 normal donors and antigen specific CD107a response to LMP1, LMP2, EBNA1.

FIG. 6b. Individual vs. Pooled LMP1, LMP2, and EBNA1 pepmix stimulation of normal donors 109 and 707 EBNA1 response. Arrow designates that EBNA1 is susceptible to competition with other pepmixes when stimulated with LMP1 and LMP2 pepmixes. LMP1, LMP2, and EBNA1 pepmixes should be pulsed individually with PBMCs rather than pooling all 374 peptides together to prevent loss of EBNA1 reactive T cells.

FIG. 6c. Donor 109 was cultured with LMP2 pepmix and cytokines. At Day 11, 79.0% of the T cell culture was recognized by the pentamer B40:01-IEDPPFNSL. High antigen reactivity was confirmed by similarly high antigen specific production of CD107a, IFNγ, and TNFα.

FIG. 6d. CD8+ T cells stimulated with LMP2 pepmix convert phenotype from CD45RA naïve cells to CD45RO Effector Memory cells. CD62L, another memory marker, as well as activation markers CD25 and CD137 are clearly upregulated between Day 7-11 of culture.

FIG. 6e. Donor 423 showed >5% to LMP2 and EBNA1 but did not respond to LMP1 pepmix.

FIG. 6f. Donor 915 demonstrates >5% antigen specific T cell reactivity to all three EBV latent proteins.

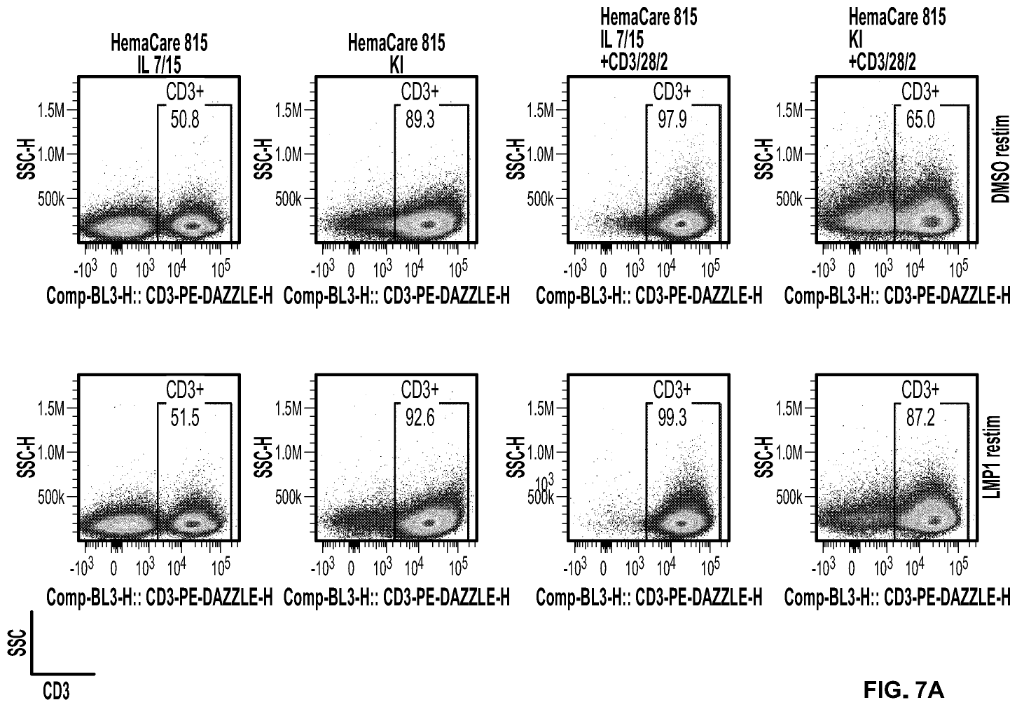

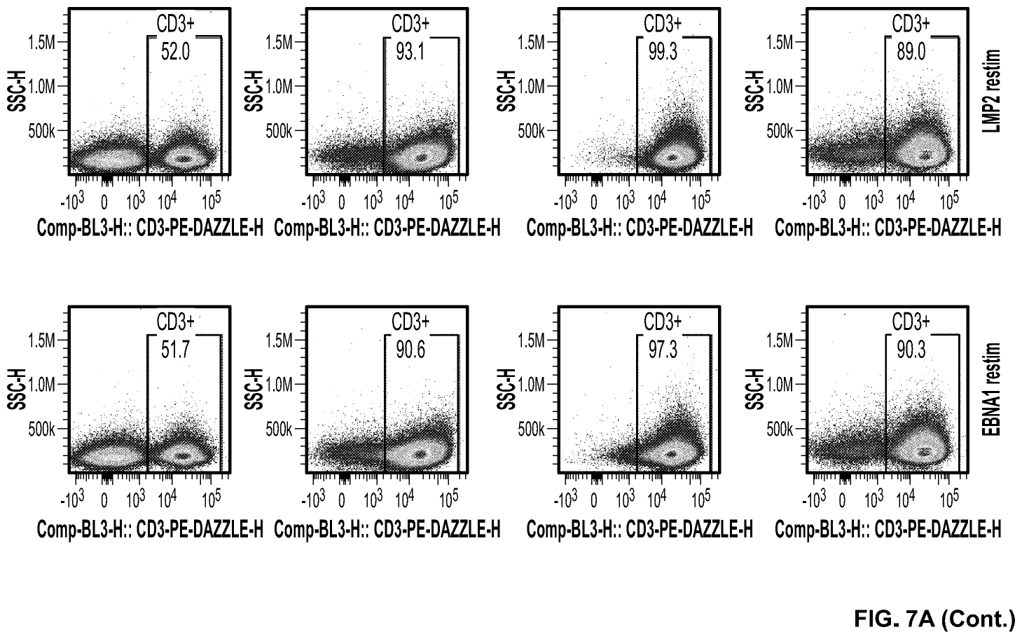

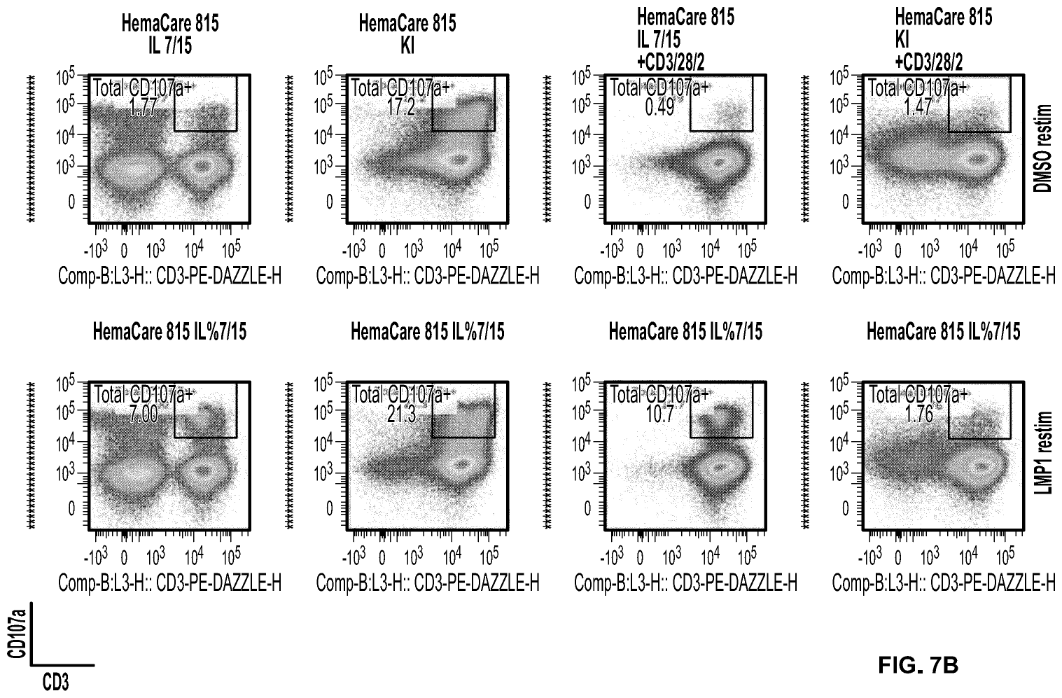

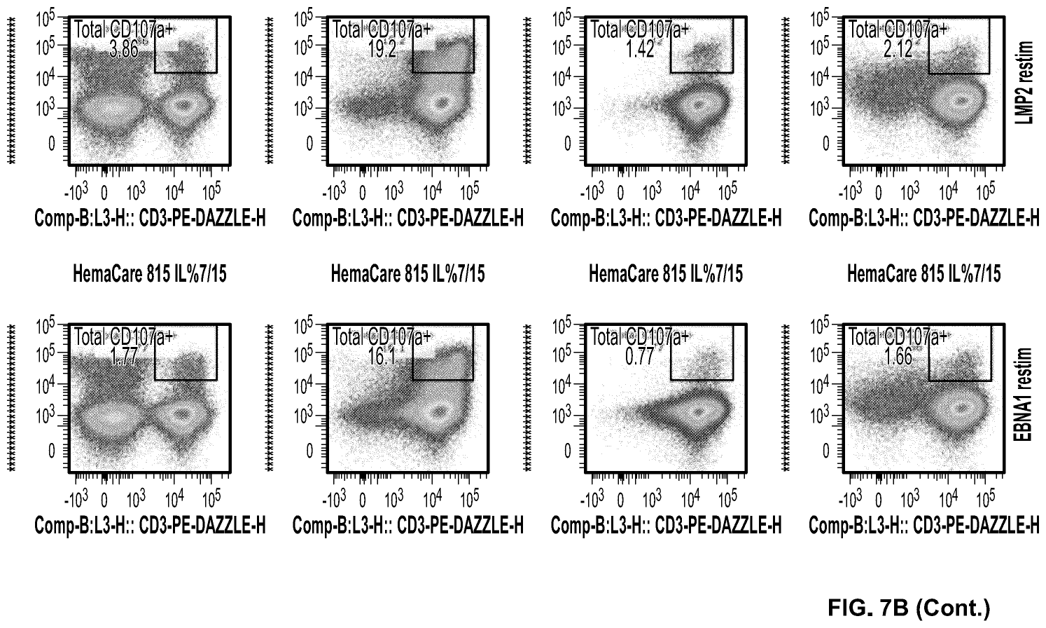

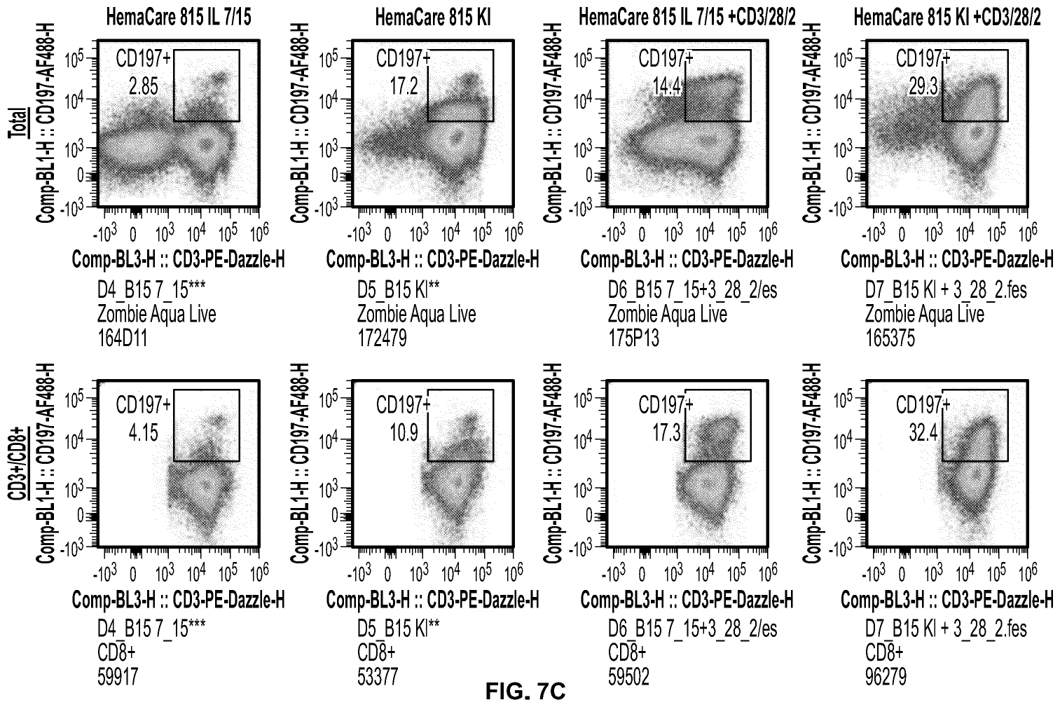

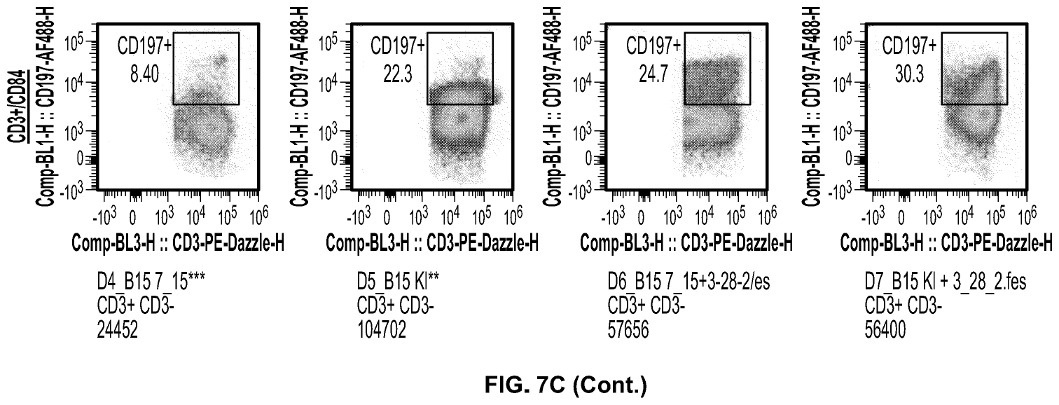

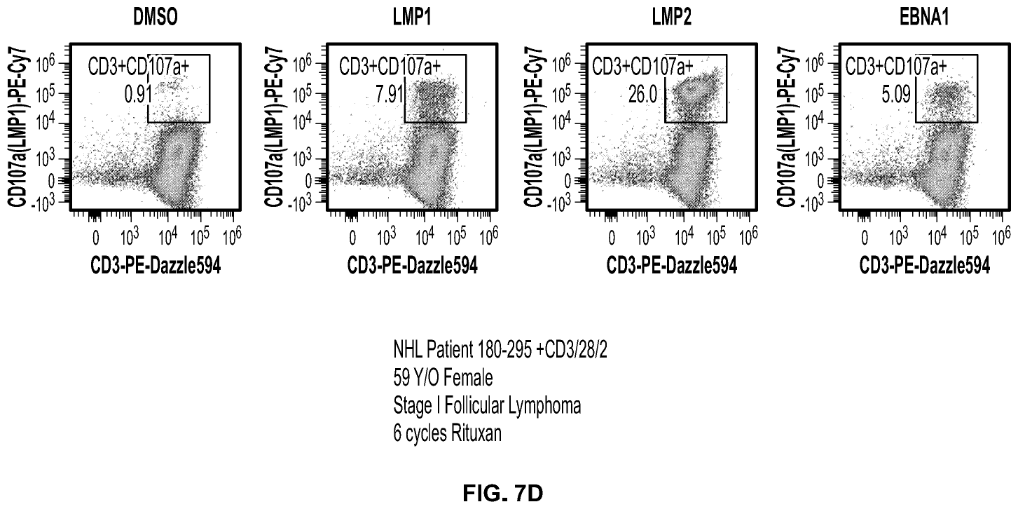

FIGS. 7a, 7b, and 7c. PBMCs from NHL patient sample HemaCare815 were expanded at research scale with cytokine combinations IL7/15 or IL2/7/15(KI) with or without CD3/CD28/CD2 polyclonal stimulation at day 14. Cells were harvested at Day 28 and evaluated for viability, % CD3 cells (FIG. 7a), % CD107a+ in response to antigen stimulation (FIG. 7b), and CD197+(memory marker expression) (FIG. 7c).

FIG. 7d. demonstrates expansion of LMP1, LMP2, and EBNA1 specific T cells from a patient with Stage I Follicular Lymphoma. Under small scale expansion conditions with IL7/15 cytokines and individual pepmix pulsing followed by pooling, the resulting cell population demonstrated >5% response to all three antigens.

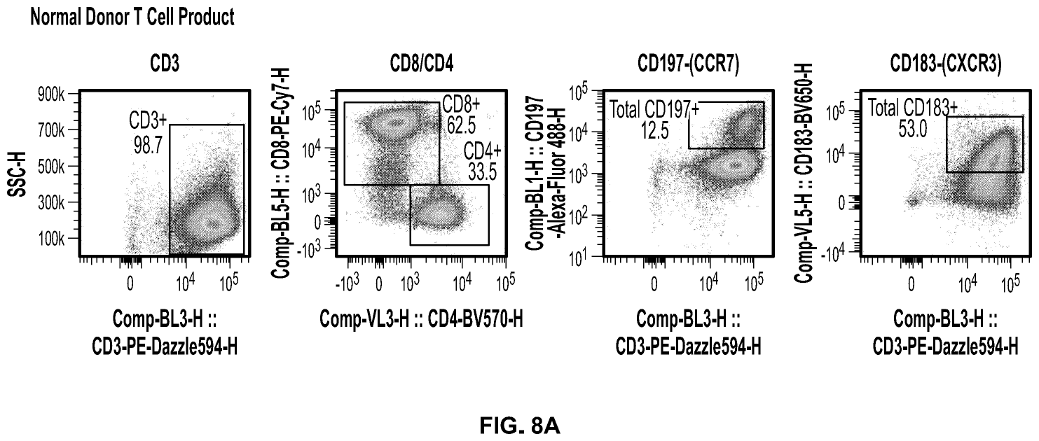

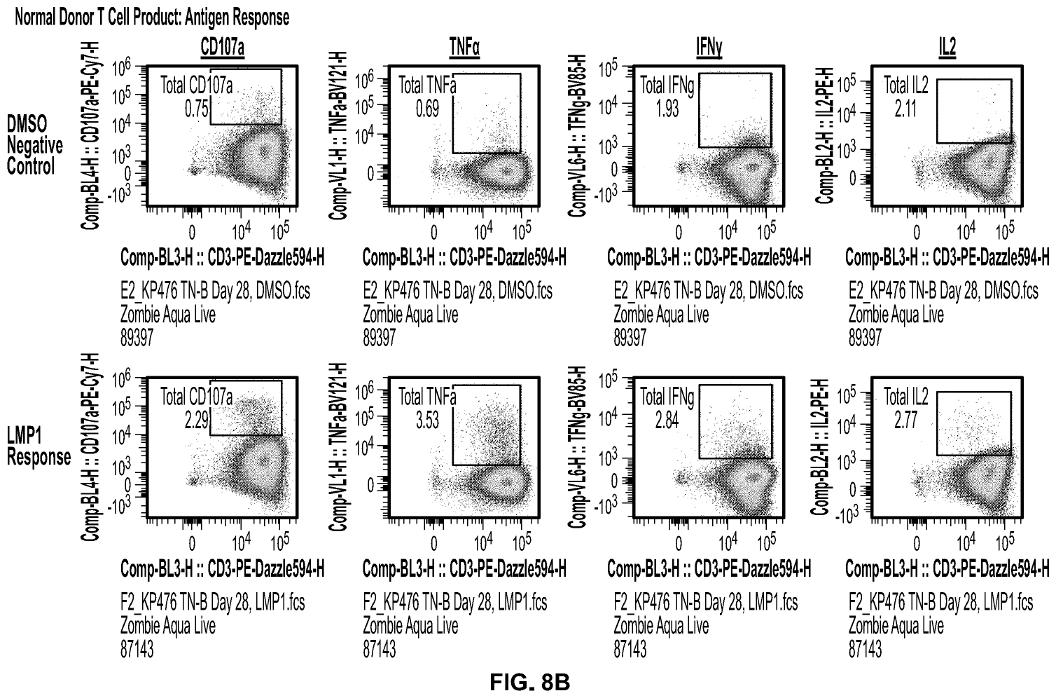

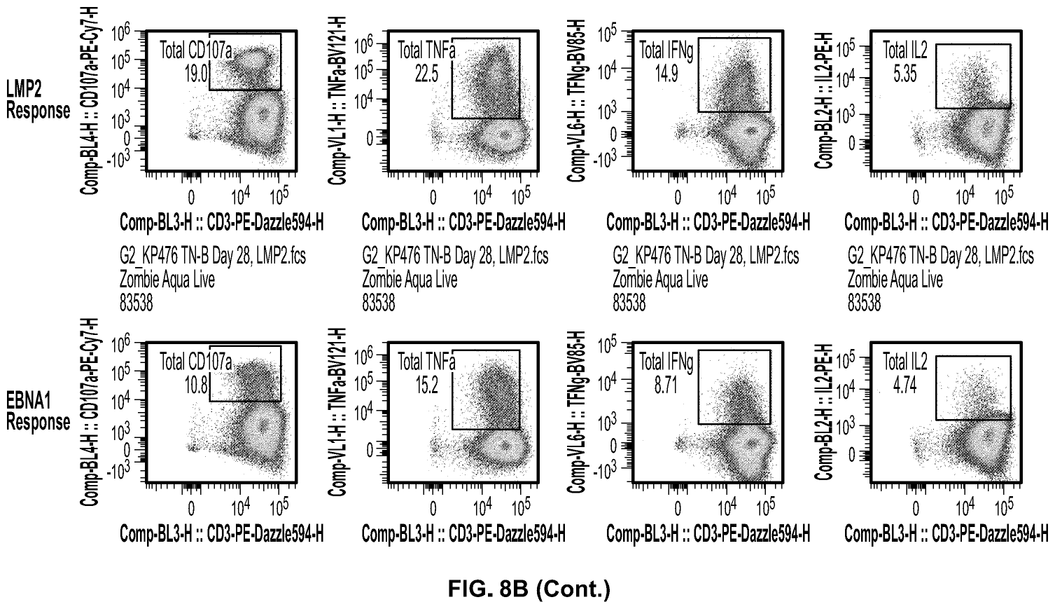

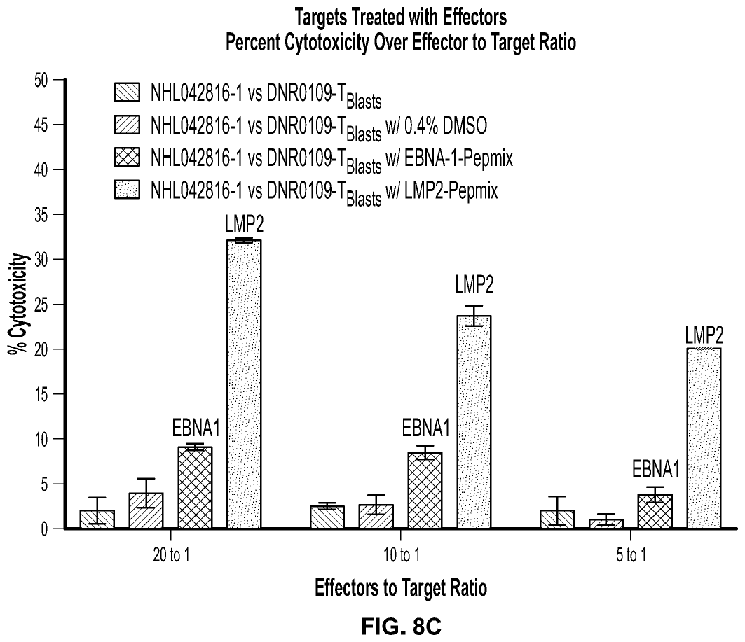

FIG. 8a [4b]. Characterization of normal donor expanded T cell product by flow cytometry. Day 28 harvest material was 98.7% CD3+ with 62.5% CD8 and 33.5% CD4. 12.5% of the CD3+ population expressed CD197(CCR7), a marker involved in homing of T cells to various secondary Lymphoid organs. 53.0% of the CD3+ population expressed CD183(CXCR3), a marker that is able to regulate leukocyte trafficking.

FIG. 8b. Characterization of normal donor expanded T cell product response to stimulation by DMSO, LMP1, LMP2, and EBNA1. The detection of CD107a degranulation, as well as TNFα, IFNγ, and IL-2 secretion follow the same ranking order of LMP2>EBNA1>LMP1.

FIG. 8c. Dose dependent selective killing of targets (T cell blasts loaded with LMP2 or EBNA1 pepmixes) by donor 109 T cell expansion product at 20:1, 10:1, and 5:1 effector to target ratios.

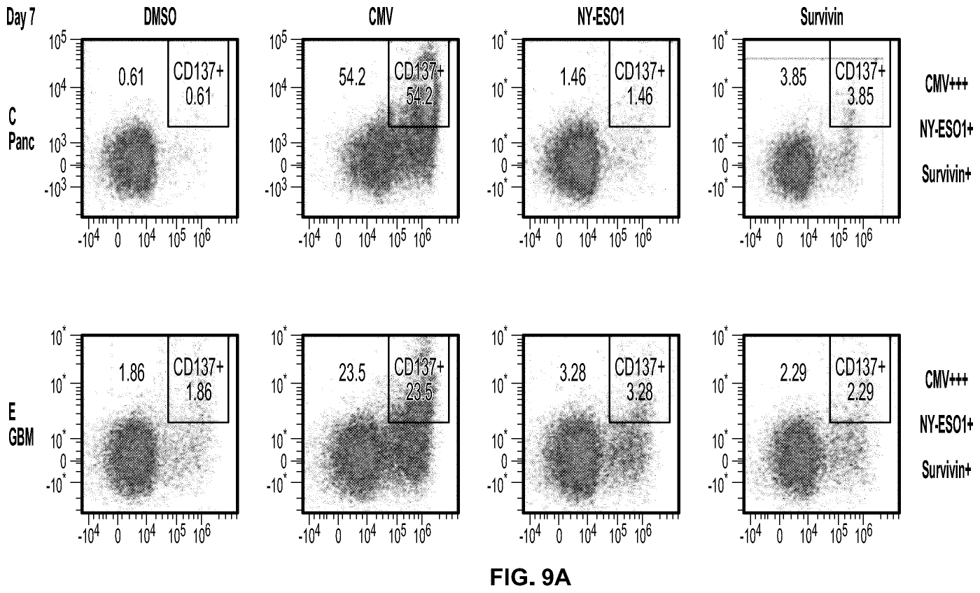

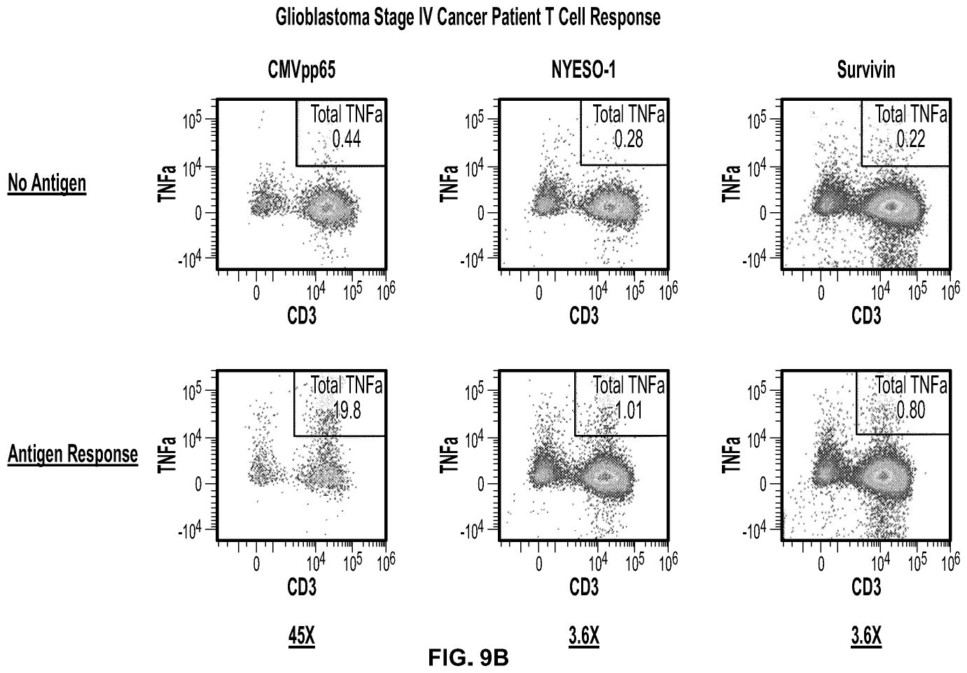

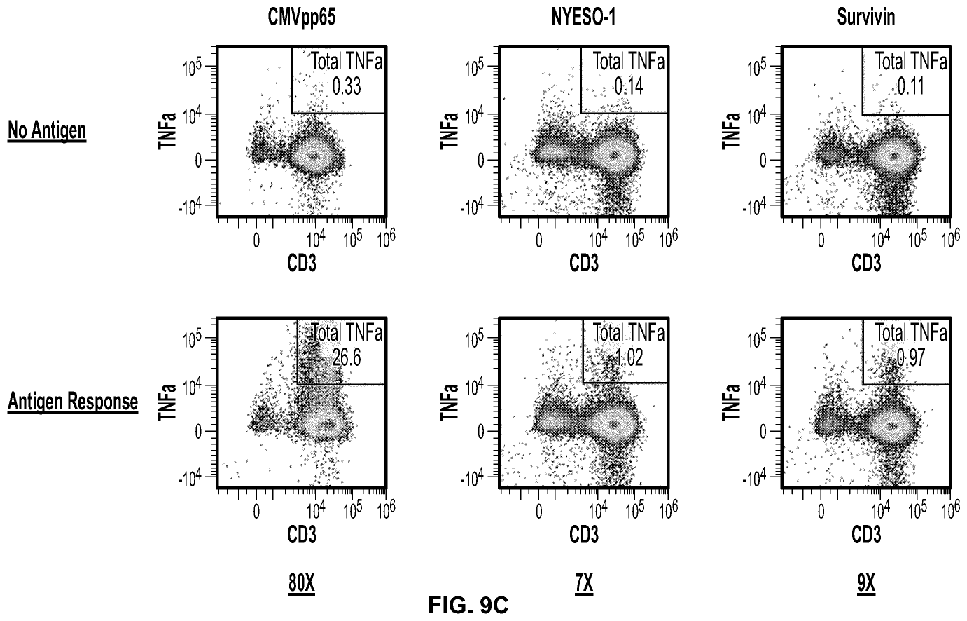

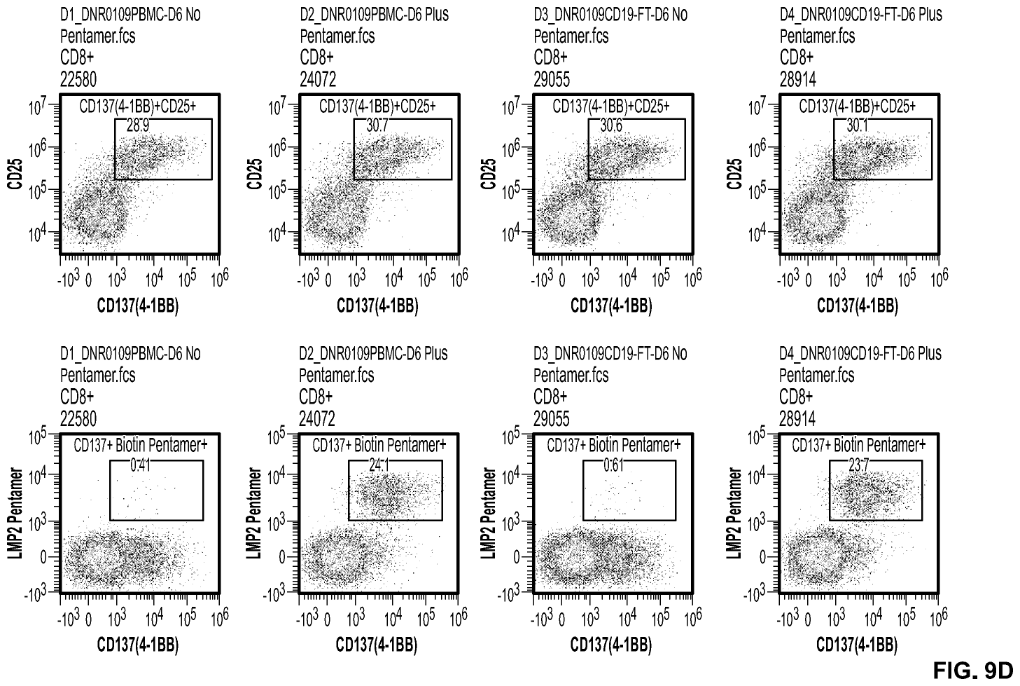

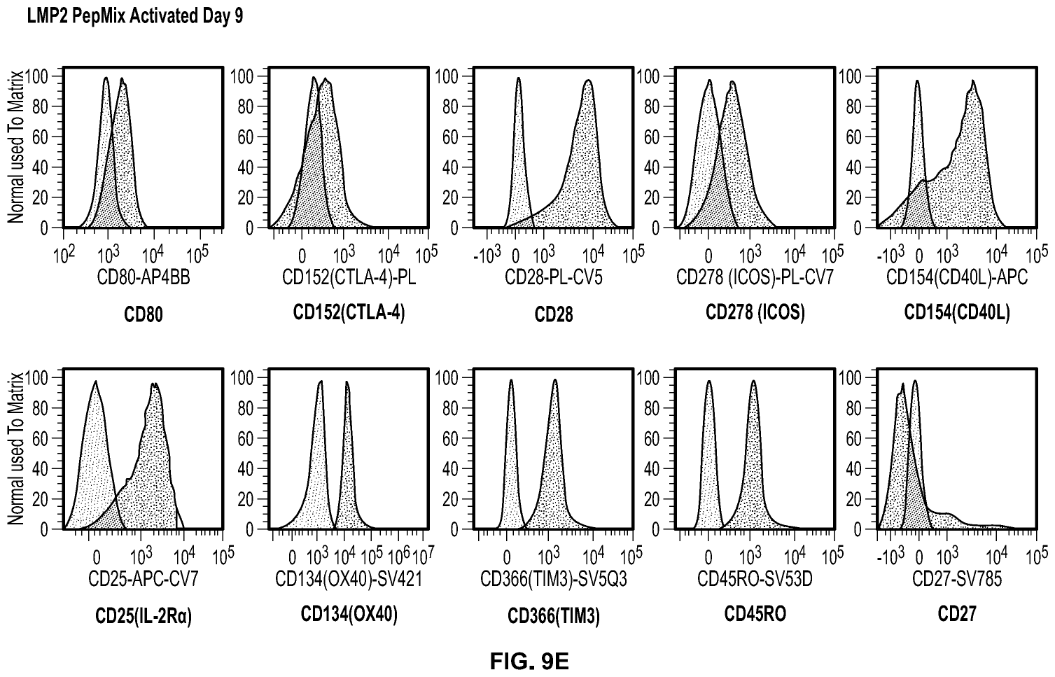

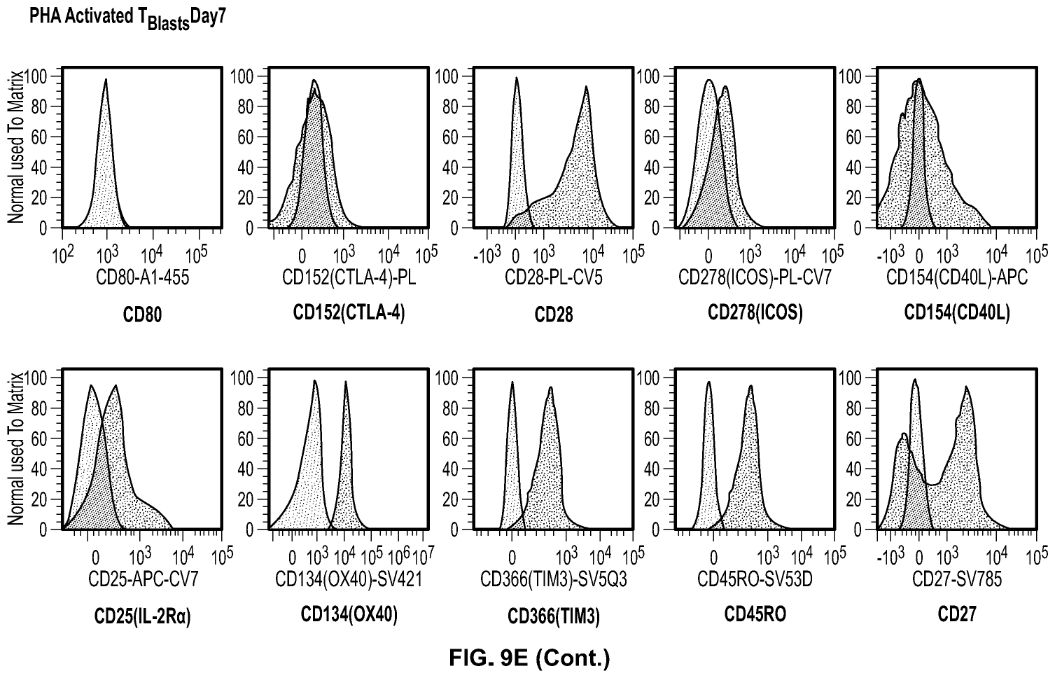

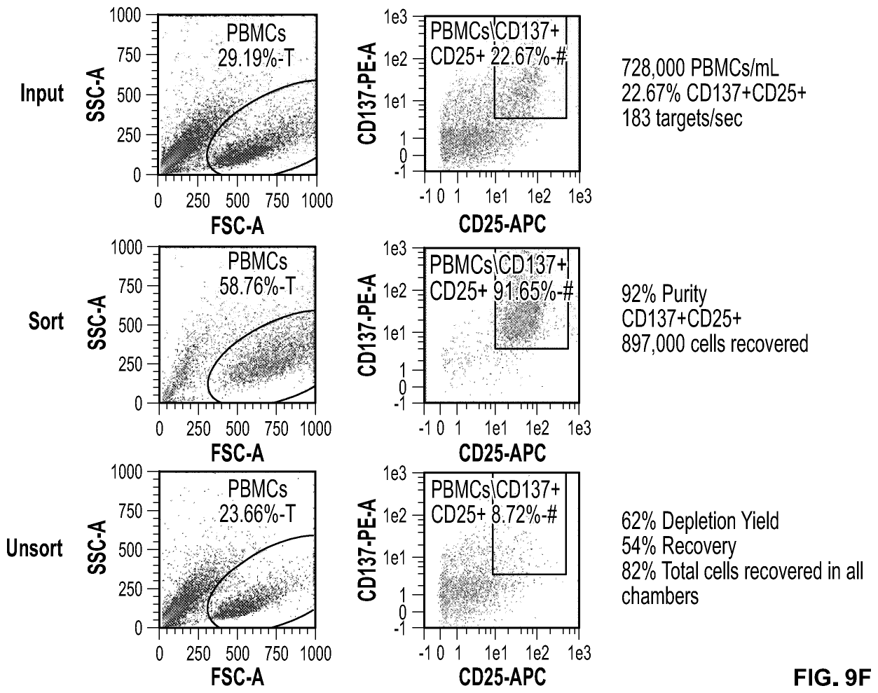

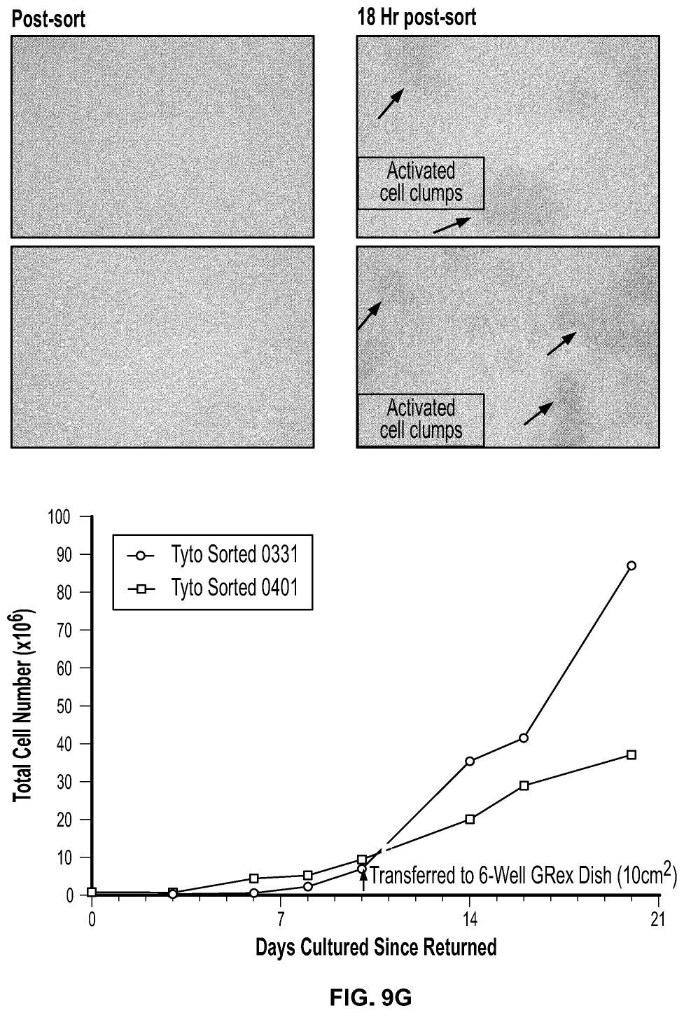

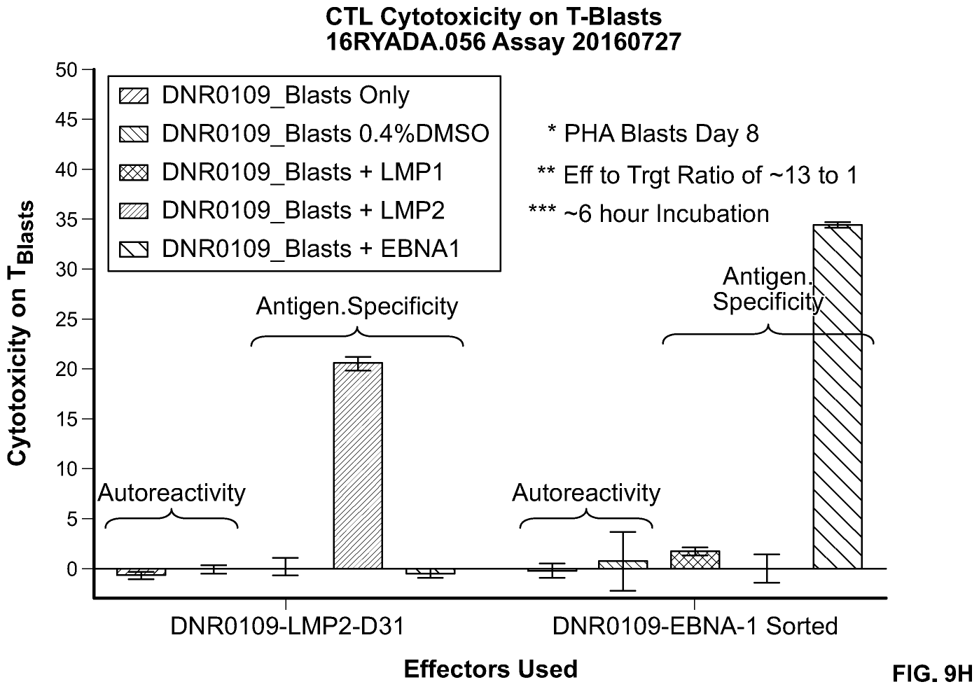

FIG. 9a. PBMCs from Glioblastoma and pancreatic patients were stimulated individual with DMSO control, CMVpp65 pepmix, NYESO-1 pepmix, and Survivin pepmix at 1 μg/ml for 7 day in culture media supplemented with cytokines (IL2, IL15, IL21). The % of activated cells specific for each antigen is listed next to the CD137+CD25+ gate.

FIG. 9b. Day 14 cultures were analyzed by intracellular cytokine staining for TNFα production in response to cellular tumor antigen is only 3.6 fold over background.

FIG. 9c. Day 14 cultures were analyzed by intracellular cytokine staining for TNFα production in response to cellular tumor antigen is only 7-9 fold over background.

FIG. 9d. Donor 109 T cells were evaluated for CD137 expression and LMP2 specific pentamer staining at Day 6 and Day. The percentage of pentamer positive CD8+ Tcells is similar to cells gated for CD137+CD25+. CD137+CD25+ markers designate an antigen activated T cell population and can be used for isolation of antigen specific T cells, either from T cell cultures or directly from patient blood.

FIG. 9e. In addition to CD137+CD25+ populations, additional activation markers on T cells could be used for isolation of antigen specific T cells. Evaluation of cell surface markers expressed on LMP2 Pepmix activated Day 9 PBMCs and PHA activated T cell blasts at Day 7. Cells were stained with the following surface markers: CD69, CD279(PD-1), CD223(LAG3), CD134(OX40), CD183(CXCR3), CD27(IL-7Ra), CD137(4-1BB), CD366(TIM3), CD25(IL-2Ra), CD80, CD152(CTLA-4), CD28, CD278(IOS), CD154(CD40L), CD45RO. Unstained cells were used as negative control and overlayed peak height of histogram plots were set to maximum. CD28, CD154, CD134, CD366, CD45RO could thus be used in addition to, or instead of, PD-1, CD137, and CD25 for isolation of activated T cells both from in vitro culture or directly from patient's blood.

FIGS. 9f and 9g. Donor 109 day 7 cultures were sorted on the Tyto (Miltenyi Biotec) with >90% purity (FIG. 9f). Sorted cells also demonstrated good viability, recovery, and morphology (FIG. 9g—morphology of recovered post-sort T cells (top panel) and culture and recovery of T cells post-sort (lower panel)).

FIG. 9h. Sorted cells (from FIGS. 9f and 9g) were expanded in media containing IL7/15 cytokines and demonstrated selective cytotoxicity against peptide loaded T cell blasts as targets.

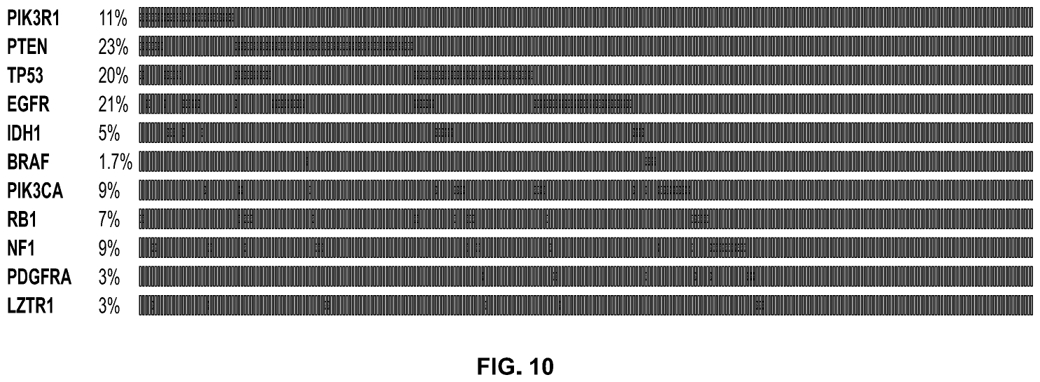

FIG. 10. Mutation frequency in the identified genes. Using standard Mutsig analysis in the above cohort 11 genes were identified in Gliablastomas (GBM) from a cohort of patients. Each column is a single patient. The second column is the frequency of the mutation in all GBM patients. For example, first patient has mutations in PIK3R, PTEN, p53 and RB.

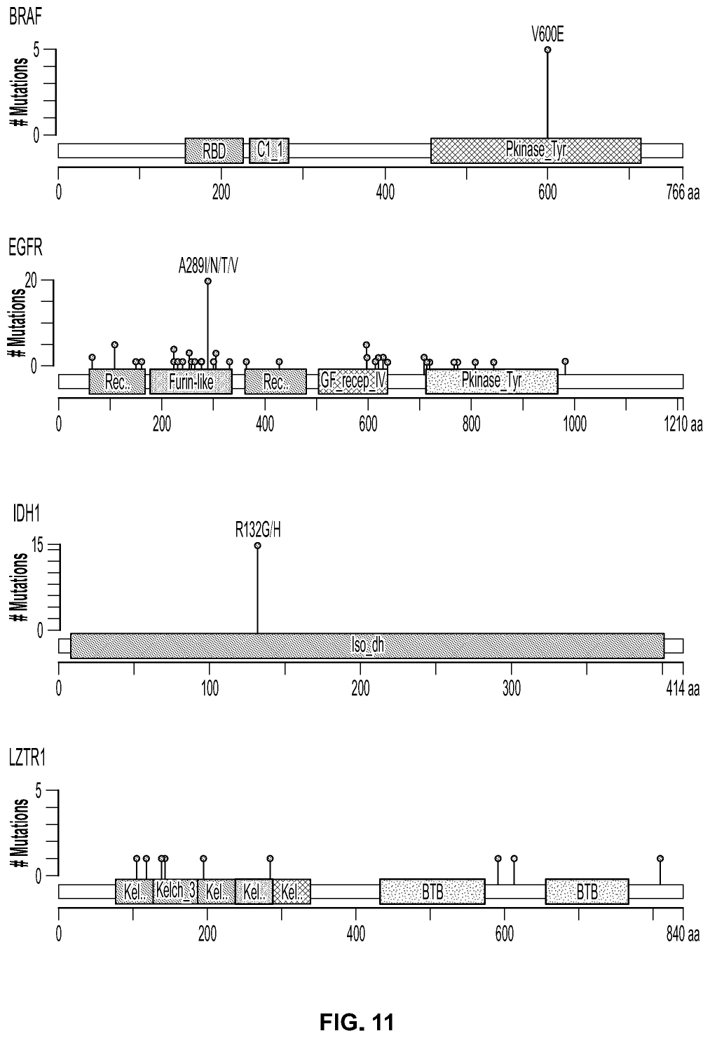

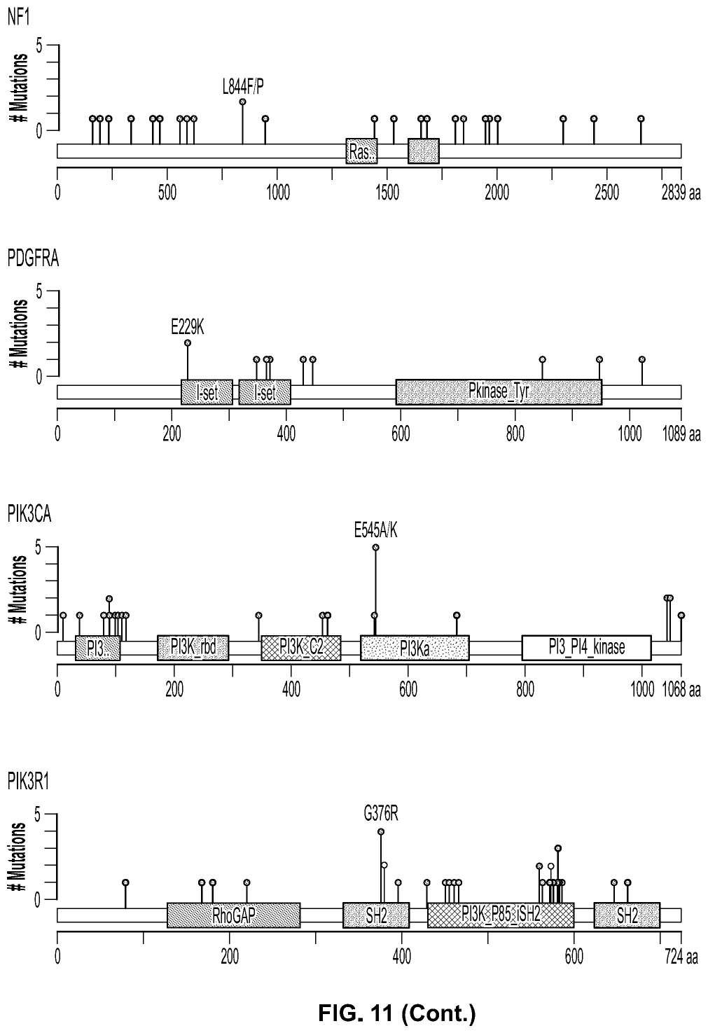

FIG. 11. Distribution of mutations in GBM patients along the selected genes.

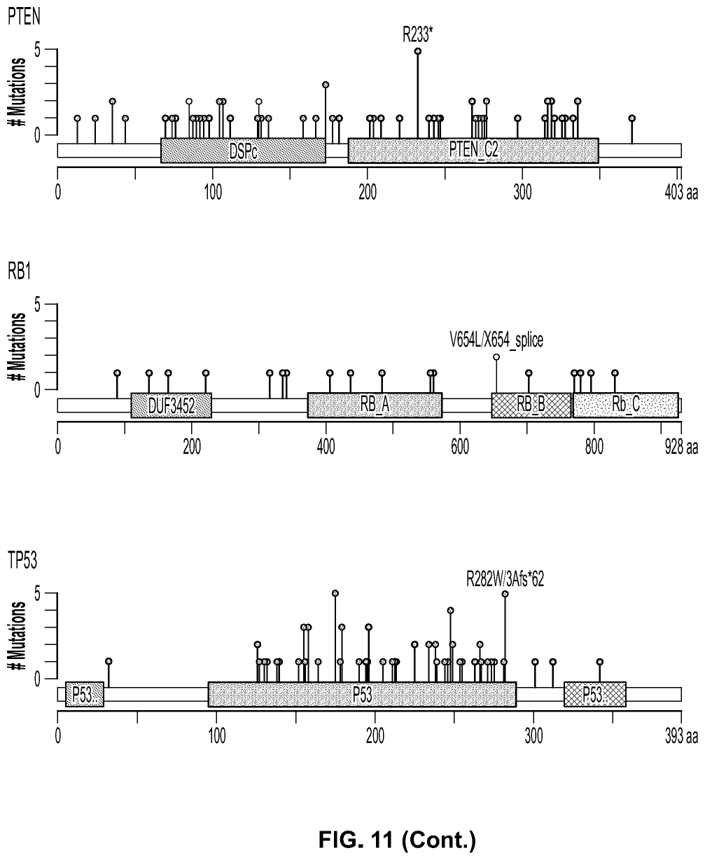

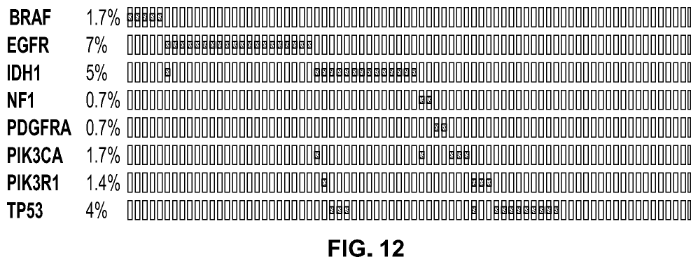

FIG. 12. Select mutation hotspots within the genes in FIG. 11. Not all hotspots are reported below as some contain stop codons. Eight neoantigens hotspots were selected with a total of 17 amino acid changes: BRAF: V600E; EGFR: A289I A289N A289T A289V; IDH1: R132G R132H; NF1: L844F L844P; PDGFRA: E229K; PIK3CA: E545A E545K; PIK3R1: G376R; TP53: R175H R248L R248W R282W. The selected neoantigens and mutational hotspots cover 58 of 291 (20%) Glioblastoma patients in the cohort and at least one binds the patient's MHC but will not generate T cells cross-reacting with wild-type protein.

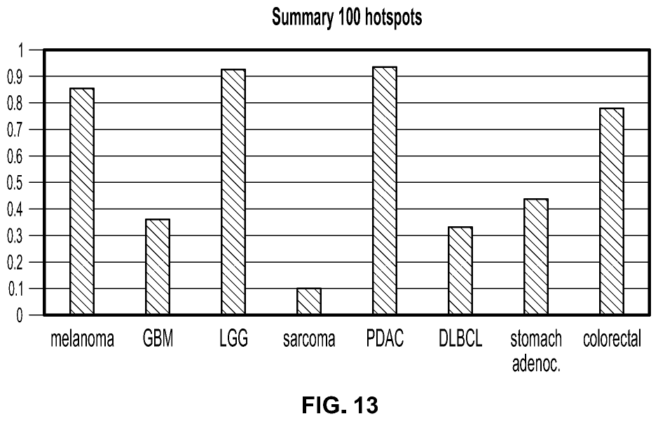

FIG. 13. Summary of the most common mutational hotspots found in human cancer was performed and the percentage of patients per cancer indication that would be targeted by these alterations is summarized verbally and graphically.

### DETAILED DESCRIPTION OF THE INVENTION

This application claims priority to U.S. Provisional Applications Ser. Nos. 62/355,458, 62/355,506, and 62/355,553, filed Jun. 28, 2016, and each is incorporated herein by reference in its entirety.

In one aspect, the present invention is directed to T cell compositions useful for immunotherapy. In embodiments of the invention, the T cell compositions are a heterogeneous population of expanded, antigen-restricted T cells. In other aspects, the present invention provides a method for creating a composition comprising T cells with specificity to one or more target antigens by expanding T cells that can bind to the target antigen(s) from a population of cells comprising T cells obtained from a patient. In certain embodiments, the cell population is sorted prior to expansion and harvesting in order to enrich for T cells that have been previously activated (either in vivo or ex vivo) by exposure to the target antigens (the “T Select” methods described herein). In other embodiments, the cell population is exposed to one or more target antigens (and certain cytokines) in order to stimulate expansion of T cells that recognize the target antigen(s) (the “T Direct” methods described herein). In embodiments of the invention, methods involving cell sorting for T cell activated in response to target antigen stimulation are performed when the reactivity to the one or more target antigens is below about 1% of the total T cell population (e.g., CD3+ cells).

Embodiments of the present invention are directed to a heterogeneous population of culture-expanded T lymphocytes, which are reactive to (i.e., restricted to) a plurality of antigens; the antigens selected based on their prevalence in patient's disease state, such that an adoptive transfer of the heterogeneous T cell population leads to the reduction or amelioration of the disease. The invention further provides methods of generating heterogeneous T cell populations. The initial T cells can be obtained from a sample of a patient's peripheral blood, bone marrow or tumor, which are then manipulated in vitro, i.e., are primed against specific antigens and then expanded with a goal to maximize the number of antigen responsive cytotoxic T cells in the final composition.

The invention provides a method of generating this heterogeneous T cell population starting from a sample of a patient's peripheral blood, bone marrow or tumor, which is then manipulated in vitro to expand T cell numbers, and where the T cells are (re)programed to become antigen-restricted. Further, the invention provides for sorting and selecting T cell subpopulations and enriching, or deleting for various subpopulations in a heterogeneous pool of cells.

T Direct Methods

In one aspect, the present invention provides a method for creating a composition comprising T cells with specificity to one or more target antigens by expanding T cells that react to the target antigen(s) from a population of cells comprising T cells obtained from a patient. A general schematic providing examples of the steps and timing for embodiments of this method for generating the heterogeneous T cells by stimulating and expansion ex vivo is provided in FIGS. 1 and 2.

In one aspect, the invention provides, a method for making a composition enriched for T cells that are reactive to one or more target antigens, the method comprising the steps of:
(a) obtaining an initial cell population comprising T-cells;
(b) stimulating the T-cells by exposing the cell population to one or more target antigens and to cytokines,
(c) culturing the cell population in media comprising cytokines;
(d) testing the cell population for antigen-specific reactivity;
(e) harvest the resulting composition comprising T cells.

In embodiments of the invention, the above method further comprises repeating step (b). In further embodiments, the above method comprises the step of polyclonal stimulation of the T cells in the cell population. In one embodiment, the initial cell population comprising T cells is peripheral blood mononuclear cells (PBMCs) from a patient's blood. In one embodiment, the initial cell population is frozen and is thawed prior to starting the method.

By this method and the variations described herein, naïve T cells and/or T cells already exposed to the target antigen(s) in vivo are obtained from the patient tissue, primed in vitro and expanded by exposure to the target antigens and certain cytokines described herein.

The specific method of T cell expansion will depend on the cell type desired in view of the particular immunotherapy useful for the disease to be treated. The cells are modified in culture by the use of agents that guide the cells towards particular phenotypes and functions. This modification is illustrated by the alteration of the physiological characteristics of the population of isolated cells from day 0 to about day 21 in culture, where the surface markers expressed by the cell population are altered and the progress of such alteration is monitored over time, as described.

T Select Methods

In one aspect, the present invention provides a method for creating a composition enriched for T cells with specificity to one or more target antigens by selecting T cells that are activated by exposure to the target antigen(s) and expanding the resulting cells. A general schematic providing an examples of the steps and timing for embodiments of this method for isolating and expanding heterogeneous T cells ex vivo is provided in FIGS. 3 and 4. In another embodiment, the invention provides a method for making a composition comprising T cells, the method comprising the steps of:
(a) obtaining an initial cell population comprising T-cells;
(b) selecting T cells based on expression of T cell activation markers,
(c) polyclonal stimulation of T cells,
(d) harvesting the resulting composition comprising T cells.

In embodiments, the method further comprises stimulating the T-cells by exposing the cell population to one or more target antigens and to cytokines. In further embodiments, step (c) is performed on the initial cell population (e.g., PBMCs). In other embodiments, step (c) is performed 6-11 days, and preferably about 7 days after stimulating the cells. In one embodiment, the method further comprising testing the initial population of cells for total T cells (CD3+), and amounts of CD8+ and CD4+ T cells, monocytes, B cells, and NK cells. In one embodiment, the initial cell population comprising T cells is peripheral blood mononuclear cells (PBMCs) from a patient's blood. In one embodiment, the initial cell population is frozen and is thawed prior to starting the method.

The T cells are selected based on expression of T cell activation markers by cell sorting or other appropriate techniques known in the art. In embodiments of the invention, the selection step is performed if the antigen reactivity of the cell population is less than about 1%. For example, on Day 7 the cells are gated on CD137/CD25 expression based on the DMSO negative control culture. For example, the GBM and pancreatic expansion had percentage of cells in this quadrant above the DMSO control. These samples are good candidates for T Select rather than T Direct. If the antigen reactivity in the cell population is sufficiently high, e.g., greater than about 1%, 2%, or 3% then the cell population can be stimulated and expanded using the T Direct methods described herein.

Target Antigens for T-Cell Stimulation

Methods of the invention involve stimulating T cells for selection and/or expansion by exposing a population of cells comprising T cells to one or more antigenic polypeptides (or other antigens) and exposing the T cells to cytokines as described herein. In certain embodiments, the antigens are one or more under-represented or non-represented antigens in subject's response to a particular disease. In embodiments, the antigens are recognized by T cells involved in a sub-dominant immune response. In embodiments, the antigens are neoantigens. In embodiments of the invention, the antigen or antigens used to stimulate T cell expansion are one or more viral proteins from cytomegalovirus (CMV), Epstein-Barr virus (EBV), hepatitis B virus (HBV), human papillomavirus, adenovirus, herpes virus, human immunodeficiency virus, influenza virus, human respiratory syncytial virus, vaccinia virus, Varicella-zoster virus, Yellow fever virus, Ebola virus, and Zika virus.

In preferred embodiments, the target antigen is presented to the cell population comprising T cells as a plurality of polypetides derived from the target antigen. The polypeptides are preferably a length suitable for efficient presentation by APCs. In embodiments, the plurality of polypeptides comprises overlapping polypeptide of 15 to 50 amino acids in length, preferably about 15 amino acids in length. In embodiments, the plurality of polypeptides comprises polypeptides that have been screened to determine antigenicity and/or dominant/subdominant status.

Certain antigens are of non-peptide origin, such as nucleic acids. Examples include RNA, such as viral RNA, CpG rich oligonucleotides, lipids, and others. Activation of intracellular recognition molecules such as Toll Like Receptors or TLRs are reported to drive T cell stimulation and proliferation.

Certain embodiments of the invention include antigens that are related to the tumor metastasis within the antigen selection repertoire. T cells generated against metastasis antigens can restrict spread of the tumor to other organs of the body. The invention includes embodiments related to immunocompetent T cell generation against metastasis antigens.

Sub-Dominant T-Cell Response(s) to Antigens

Antigens useful in the methods of the invention are identified based on a number of approaches. U.S. application Ser. No. 14/122,036, incorporated herein by reference, details the use of subdominant antigens to reprogram the immune response.

Cancer Antigens—Viral Proteins

Certain virus proteins are associated with, and expressed in, particular types of cancer. Epstein-Barr virus (EBV) is one of the most common viruses in humans and is associated with lymphoma (Hodgkin's lymphoma, Burkitt's lymphoma and conditions associated with human immunodeficiency virus (HIV), such as hairy leukoplakia and central nervous system lymphomas), gastric cancer, and nasopharyngeal carcinoma. EBV becomes latent in certain cell types that it infects, for example, B cells. Even when latent, EBV expresses certain proteins that can be targeted by the methods of the invention in order to generate an expanded T cell population with T cells that recognize one or more EBV proteins and therefore can be used in adoptive therapy generating an immune response to cells in which the EBV proteins are expressed. In one embodiment, the EBV antigens used to stimulate and thereby expand T cells is one or more of LMP1, LMP2, EBNA1, and BZLF-1. In other embodiments, EBV Latency III proteins are target antigens. In one embodiment, the antigens used to stimulate T cells are a plurality of polypeptides derived from one or more of LMP1, LMP2, and EBNA1.

The T cell compositions made by methods of the invention using EBV latent proteins (e.g., LMP1, LMP2, and/or EBNA1) as a source of antigens are useful in treating diseases where targeting a subject's immune system to cells or tissues expressing those proteins is beneficial. A variety of cancers such as non-Hodgkin's lymphoma (NHL), gastric cancer, and nasopharyngeal cancer are often characterized by expression of latent EBV proteins. Accordingly, the aspects of the invention relate to treating such cancers by administering to a patient a T cell composition generated by the methods of the invention to expand T cells that recognize latent EBV proteins (e.g., LMP1, LMP2, and/or EBNA1).

Likewise, CMV proteins, are expressed in certain cancers such as glioblastoma, glioma, colon, salivary gland cancer. In one embodiment, the methods of the invention, are used to generate a T cell population enriched in T cells that recognize CMV antigenic proteins. In one embodiment, the CMV antigen used to stimulate and thereby expand T cells is pp65. Cancer/testis antigen 1 (NY-ESO-1) and Survivin, like pp65 are expressed in glioblastomas. In one embodiment, the antigens used to stimulate T cells are a plurality of polypeptides derived from one or more of pp65, Cancer/testis antigen 1 (NY-ESO-1) and Survivin.

Cancer Antigens—Overexpressed Antigens

Target antigens that can be used to stimulate and expand T cells in the methods of the invention to generate compositions enriched for T cells reactive to the target antigen include antigens that are overexpressed or mis-expressed in particular cancers. Examples of such cancer-associated antigens is Cancer/testis antigen 1 (NY-ESO-1) and Survivin in glioblastoma.

The antigen PSMA is found on healthy tissue but is upregulated in prostate tumors and highly upregulated in metastatic tumors, and accordingly, may be used as a target antigen in methods of the invention.

Cancer Antigens—Neo-Antigens

In one aspect, the invention relates to the selection, production and use of neoantigens, that provide for generating novel immune modulating therapeutics. In certain embodiments, the invention described herein relates to neoantigen compositions, and methods of generating compositions comprising T cells that are neoantigen restricted.

A “neoantigen” as used herein in an antigenic polypeptide that is absent from the normal/naïve human genome, but is present in a cancer cell due to mutation, rearrangement or epigenetic changes. Thus, neoantigens are tumor-specific antigens (TSAs).

Neoantigens may be used in the methods of the invention to produce neoantigen-reactive T cell populations that are useful in adoptive therapies for the treatment of, e.g., cancer. In addition to the approach of reprogramming the antigen-specificities of the immune response away from dominant antigens that induce tolerance toward subdominant antigens, the present invention provides neoantigen compositions that serve as alternative antigen targets, toward which the immune response can be directed. These neoantigens may already reflect subdominant antigens within a patient's immune response, or they may not be represented in the T cell repertoire of a patient. The use of neoantigen-reactive T cells are not generally affected by central T cell tolerance, as would be the case for self-reactive antigens and some tumor-associated antigens, which make these cell preparations highly desirable as therapeutic agents and vaccines.

In the methods described herein, useful neoantigens are tumor specific antigens which may be universal to that tumor type or may be patient-specific and tumor-specific neoantigens; the differences being in the expansion and selection of the neoantigen-reactive T cell populations, not selection of the neoantigens. Briefly, T Direct employs an antigen edited T Cell technology, to prime and expand T cells to multiple neoantigens relevant to cancers. Administration of these T cells to the patient creates a new immune response, effectively targeting the tumor with T cells reactive to multiple antigens. T Select utilizes PBMCs to select tumor activated T cells from blood, with specific reactivity towards multiple neoantigens. This method provides a T cell therapy personalized for each patient's tumor. T Select allows neoantigen T cell therapy to be practical, with no need to pre-identify and synthesize personal neoantigen peptides for each patient.

Neoantigens are determined as suitable for the invention in a first aspect by analyzing a disease state and identifying an antigen that is present in the disease but preferably not in healthy tissues, e.g., as a result of cellular mutations. For patient-specific approaches, the patient's immune response may be biased to specific dominant antigens, as can determined by epitope mapping, which should be avoided when selecting neoantigen candidates for immune stimulating effects, but may be useful when selecting candidates for immune attenuating effects. A preferred neoantigen is an antigen that is associated uniquely with the disease state, but also suitable are a tumor associated antigens that are upregulated in the disease state. Thus, BRCA2 mutations, EGFR mutations such as EGFR L858R, ALK gene fusions, ROS1 gene fusions, BCR-ABL1 fusions, BRAFV600E, TP53 R273H and similar mutations all provide excellent neoantigen candidates.

High affinity T cells specific only for tumor antigens are a useful source of neoantigens. As a tumor grows and evades the immune system, it typically accumulates genetic mutations. Certain cancers such as lung, bladder, breast cancer and melanoma may contain 500 or more mutations. There are specific genomic loci known to be mutated frequently in various cancers, referred to commonly as “hotspots”, such as the KRAS gene mutations observed commonly in colon and lung cancers and other “long tail” hotspot mutations in various oncogenes. Other genes with focused mutational hot spots include BRAF, seen in 50% of melanoma patients with 90% of these mutations being V600E; BCR/Abl translocations, seen in 95% of CML patients, IDH1 seen in 70%-90% glioma/glioblastomas patients, and p53 mutations seen in many cancers.

The advent of high throughput massively parallel sequencing (“next-generation sequencing” or NGS) provides an effective way to discern a large amount of genomic information. Mutations in tumor surface antigens relative to wild-type provide useful candidates for reference antigens because these are tumor specific. Gene sequence information from a patient provides for a baseline from which mutations can be assessed, and is useful in connection with the T-Direct and T-Select modalities described in our related applications. Sequencing of tumors or diseased tissues permits identification of gene mutations at hotspots. Known cancer genes (“gene panels”), whole-exome, whole-genome and/or whole-transcriptome approaches provide useful ways to detect cancer mutations and therefore to develop customized immune therapies targeting the tumor. For example, NGS permits subtractive genetic analysis, e.g., sequencing a primary tumor and metastatic tumors for determining genetic differences, or sequencing a patient tumor for comparison to reference sequences, or sequencing a patient's tumor genome for comparison against a genetic readout of their noncancerous tissue. Mutations in exposed epitopes of an antigen are particularly good neoantigen candidates. Tumor specific (somatic) mutations, copy number changes and translocations are identified by next generation sequencing (frozen or fixed tumor vs normal tissue). Somatic tumor specific mutations and translocations translate into shared tumor specific neoantigens. Copy number changes translate into tumor associated neoantigens. By combining T Direct and T Select with these diagnostics, we create a system to rapidly create customized T cell therapies to neoantigens in a practical way. Tumor cells also extravasate into the blood enabling detection in circulating DNA. Combining neoantigens obtained from blood with T Direct and T Select creates a complete system to identify neoantigens and source T cells for production of expanded T cell therapies from blood samples. This can be accomplished with a single draw from a patient, enabling customized “one-stick” therapeutics that can evolve over time, or can be derived from archived blood.

Sequencing a person's genome for highly specific personal mutations significantly increases the chances of obtaining unique neoantigens, against which highly effective T cells could be generated. On the other hand, the cost of individual genome sequencing prior to designing an effective therapy is not cost effective. Hence, alternative strategies include shared tumor-specific neoantigens (shared by tumors rather than unique to each patient), which may be targeted as efficacious neoantigens using knowledge gained in genomic tumor evolution models. Point mutations, which are unique to a cancer subclass or a common cancer evolutionary trunk, i.e., “driver mutations” and “trunk antigens” (i.e., on a phylogeneic map), providing excellent selections for generating neoantigen restricted T cell populations. Genomic evolution studies between primary and metastatic tumors are useful to select mixtures of neoantigens for raising immune responses for adoptive T cell therapy. By targeting common mutations in the trunk of tumor evolution, one may eliminate the primary tumor and any recurrence.

Pre-identification of the neoantigens is not necessary when using T cells obtained from blood, which will be reactive to antigens on primary tumors and metastases, as opposed to TILS which will be reactive to antigens found within the tumor. Neuroblastoma, colorectal, ovarian, breast, melanoma and hepatocellular cancers are most amenable to selecting shared tumor specific neoantigens and growing reactive T cells from blood (all >1000; >70% ctDNA) followed by bladder, gastroespohageal, pancreatic, head and neck cancers (all >500; >70% ctDNA). Bettegowda et al. Sci Transl Med (2014) 6(224):224 (incorporated herein by reference) provides for the frequency of detectability of neoantigens from tumors in blood. Blood provides for a novel proprietary system for treating cancer and serious diseases, in a direct pathway from the patient, to the lab, (back) to a/the patient. By obtaining blood, it is possible to educate T cells to seek and destroy the patient's tumor—both hematologic and solid malignancies.

In various embodiments, a neoantigen is not present in a target tissue but is introduced to a tissue that will be targeted for an antigen-restricted immune response. For example, a neoantigen from an oncolytic virus is added to the tumor by infection of the tumor. In particular, Lassa-VSV targets cancer cells in brain after intravenous or intracranial injection, such as glioma. Lassa-VSV also targets melanoma and ovarian cancer. It infects metastasizing cancer cells without infection of normal cells. Lassa-VSV generates strong immune responses, particularly T cell responses, and generates high affinity antibodies to multiple antigens from infected cells. A Lassa-VSV-restricted T cell transplant provides for increase in survival of cancer-bearing (GBM) animals indefinitely, appears to eliminate chemoresistant cancers, and appears to completely eliminate some cancers. Therefore, according to the invention a preparation of Lassa-VSV is introduced to the tumor, and a Lassa-VSV-reactive T cell preparation is provided subsequently, which targets and clears the infection thereby reducing the tumor burden.

Other antigen markers of disease associations are described in the scientific and medical literature, and the invention described herein is not intended to be limited to only classical neoantigens, or only those specific neoantigens identified, or solely the antigen types or disease states specified. The choice of neoantigen is motivated by the specific type of immune response modulation desired, in view of the disease state to be treated, such as would be apparent to one of skill in the art. Furthermore, neoantigen selection may be guided by identification of particular epitopes that can be validated and optimized for their T-cell reactivity.

Neoantigens are useful to modulate (i.e., either upregulate or tolerize) a specific immune response. A given candidate neoantigen being selected as described herein, may be used directly or may be modified further by common methods known in the art, including amino acid mutagenesis, cyclization, glycosylation or other chemical modifications, such as including the addition of haptens. For example, the neoantigen candidate may be modified by amino acid replacement, to produce a peptide that binds MHC class I structures with higher affinity.

A validated neoantigen is described as being associated with a disease state that is amenable to immune therapy, and where the neoantigen is capable of binding to MHC class I and/or class II molecules, and is immunogenic to T cells in that it causes T cell activation, proliferation and/or memory responses in CD4+ and/or CD8+ subpopulations. Preferably, a validated neoantigen is also subdominant in the target patient. More preferably, one or more validated neoantigens are used to induce an immune response in a heterogeneous pool of T cells. In various other embodiments, three or more neoantigens are prepared and validated. The number of neoantigens in the preparation used to immunize T cells may include ten, fifteen or twenty or more individual neoantigens. The immunogenicity of various neoantigens will not be equal, and so the immunization protocol can be designed to avoid creating dominant responses.

Ras is a family of structurally related small GTPase proteins, which are expressed in all cells, and are involved in the regulation genes involved in cell growth, differentiation and survival. Mutations in three Ras genes (HRas, KRas, and NRas) are the most common oncogenes in human cancers and cause uncontrolled proliferation. Ras mutations are found in 20% to 25% of all human tumors, and up to 90% in certain types of cancers.

Constitutively activated Ras can contain one or more mutations that eliminate or reduce GTP hydrolysis, which results in the protein being rendered permanently active. The most common Ras mutations are found at glycine residue G12 within the P-loop, as well as the catalytic residue Q61. A glycine to valine mutation at residue 12 renders the GTPase domain of Ras insensitive to inactivation by GTPase activating proteins and thus constitutively active. The glutamine at residue 61 stabilizes the transition state for GTP hydrolysis, and mutation of Q61 to lysine effectively eliminates hydrolysis. Other important mutations include S17N and D119N.

In accordance with the invention, Ras-based neoantigen candidates are designed and validated as follows. A portion of a patient's genome including Ras is sequenced and the patient's tumor is sequenced, or a consensus tumor sequence is derived, and differences between the two are ascertained. The above Ras mutations are typical of expected sequencing results, and provide excellent neoantigen candidates. Peptide sequences of approximately 8-10 amino acids in length are created, spanning the mutation sites (i.e., at the first, second, third etc. up to eighth amino acid position). These candidate peptides are evaluated for potential MHC class I binding fit by computer modeling. Best fit candidates are advanced. These sequences are extended up to 15-24 amino acids in length using the tumor sequence. These longer peptides are modeled for class II binding fit, and optionally their ability to bind MHC class II is validated empirically. Peptide sequences that are able to bind MHC class I and/or class II structures are used to prime T cells as described in our related applications. In specific embodiments, the MHC haplotypes CW8, A3 and A68 are preferred. In total, these MHC alleles are represented in about 40-50% of patients. These HLA types can bind long peptides containing KRas point mutations specifically, while they do not bind normal Ras sequences. These HLA types are positive with IFNg/TNF alpha ICS and CD107a stimulation. With these MHC alleles, the response to KRas does not run a risk of autoreactivity to the wild-type Ras sequence, while KRas is mutated in 90% of pancreatic cancers, 30-60% colon cancers, and 20-30% in lung adenocarcinoma. In other embodiments, T cells obtained from blood are screened against panels of Ras peptides, and the reactive populations amplified.

The T cell receptors from neoantigen-reactive stimulated CD8+ and/or CD4+ T cell, selected from cells in an immunized cell population, are useful for neoantigen validation since such a reactive T cell it is highly dispositive of immunogenicity. The T cell receptors may be sequenced and cloned, for example by PCR. See, Boria et al, Primer sets for cloning the human repertoire of T cell Receptor Variable regions, BMC Immunol. 2008; 9: 50; Guo, et al., Rapid cloning, expression, and functional characterization of paired αβ and γδ T-cell receptor chains from single-cell analysis, Molecular Therapy—Methods & Clinical Development 3, Article number: 15054 (2016); see also Simon et al., Functional TCR Retrieval from Single Antigen-Specific Human T Cells Reveals Multiple Novel Epitopes, Cancer Immunol Res December 2014 2; 1230. TCRs can be cloned into a number of suitable vectors, including those containing sequences for transfection. In certain embodiments, a preferred vector has integration sequences for introducing as a transgene, the cloned TCR sequence into a target T cell. In various embodiments, neoantigens are used to raise T cell responses; the CD8+ and CD4+ populations are sorted and screened for neoantigen reactivity, and such cells are panned further for highly immunogenic subpopulations, where the T cell receptor sequences are sequenced and cloned. In certain embodiments, a TCR from a neoantigen-restricted T cell is cloned into a memory cell. In other embodiments a TCR from a neoantigen-restricted T cell is cloned into a Treg.

Chimeric Antigen Receptor T Cells (CARTs) are generated by linking the variable regions of immunoglobulin heavy and light chains to the intracellular signaling chains in the T cell receptor. CARTs are not restricted to interactions with MHC structures for activation. See Pule, et al., Virus-specific T cells engineered to coexpress tumor-specific receptors: persistence and antitumor activity in individuals with neuroblastoma. Nature Medicine 14, 1264-1270 (2008); see also Davila et al., Efficacy and Toxicity Management of 19-28z CAR T Cell Therapy in B Cell Acute Lymphoblastic Leukemia, Sci Transl Med. 2014 Feb. 19; 6(224). For further background, see Dotti, et al., Design and Development of Therapies using Chimeric Antigen Receptor-Expressing T cells, Immunol Rev. 2014 January; 257(1): 10.1111/imr.12131. See also, Kochenderfer J N, Rosenberg S A., Treating B-cell cancer with T cells expressing anti-CD19 chimeric antigen receptors. Nat Rev Clin Oncol. 2013 May; 10(5):267-76, which concludes that the potent antigen-specific activity of CARTs observed in patients suggests that infusions of anti-CD19 CART cells might become a standard therapy for some B-cell malignancies. Accordingly, in certain embodiments the invention provides a CART population directed to a neoantigen. To generate such a CART, antibodies specific to a neoantigen provide the source of Ig heavy and light chains used to create the targeting component of the CART. Such antibodies may be raised by immunization and selection methods, or may be generated by cloning or synthesized from sequence information.

Validation of Antigens

Methods of the invention involve stimulating T cells for selection and/or expansion by exposing a population of cells comprising T cells to antigenic polypeptides (or other antigens). In embodiments of the invention, an antigen is validated by confirming its immunogenicity. The immunogenicity of an antigen, i.e., the ability of an antigen to trigger an immune response, depends largely on its presentation to T cells by the numerous types of antigen presenting cells (APC) such as but not limited to dendritic cells (DC). APCs typically display major histocompatibility class (MHC) structures (class I and class II) in the context of which an antigen is displayed. Validation of antigens in view of the above is accomplished by determining if the antigen provides a suitable fragment for binding MHC structures, that is, the antigen is capable of binding to MHC class I and/or class II molecules, and is immunogenic to T cells in that it causes T cell activation, proliferation and/or memory responses in CD4+ and/or CD8+ subpopulations.

MHC class I molecules (HLA-A, B, C, E, F and G) display peptide fragments of antigen proteins to CD8+ cytotoxic T cells, which triggers a direct response from the T cell against a target antigen. MHC class II molecules (HLA-DM, HLA-DO, HLA-DP, HLA-DQ and HLA-DR) are found on APC such as DC, mononuclear phagocytes, certain endothelial cells such as thymic epithelial cells, group 3 innate lymphoid cells and B cells. MHC class II molecules display peptide fragments of neoantigen proteins to CD4+ helper T cells, which trigger various immune responses such as activation of B cells and the humoral response, inflammation and swelling due to recruitment of phagocytes, as well as long-term immunological memory. Functionally, MHC class II molecules present extracellular antigens (unlike class I molecules, where the antigen is cytosolic, such as a viral peptide antigen). One object of the invention is to reprogram the natural immune responses through the use of antigens, and accordingly, the present techniques can provide means for triggering e.g., cytotoxic T cell responses to typically extracellular antigens, and/or helper T cell responses to typically cytosolic antigens. To accomplish this, an antigen can be validated for its MHC class I and class II binding ability.

MHC class I molecules are heterodimers, having a α chain and a β2-microglobulin (b2m) light chain, linked noncovalently through interactions of b2m and the α3 domain. The α chain is polymorphic and encoded by an HLA gene, while b2m is ubiquitous. The α3 domain spans the plasma membrane-spanning and interacts with the CD8+ co-receptor, which stabilizes the interaction between the T cell receptor (TCR) and the MHC class I molecule, at the α1-α2 heterodimer. The α1 and α2 domains fold to make up a groove for peptides 8-10 amino acids in length. The TCR mediates a determination of antigenicity for the neoantigen fragment held in the groove.

MHC class II molecules are heterodimers of two homogenous peptides, an α and β chain. The antigen-binding groove of MHC class II molecules is open at both ends in contrast to the corresponding groove on class I molecules, which is closed at each end. Accordingly, the neoantigens presented by MHC class II molecules may be between 15 and 24 amino acids in length. MHC class II bind to CD4 as well as a number of other cellular receptors on T cells and DC (such as LAG-3).

There are numerous tools that can be used to aid in a determination that a candidate will bind in the MHC class I peptide groove. There are structural data sets in the scientific literature where binding parameters are visually described, for various class I and/or class II-antigen complexes. In addition, the parameters of the grooves and floors of the binding pockets have been resolved through mutagenesis techniques, and so much is known about the molecules and their antigen binding parameters. Accordingly, there are bioinformatics-based predictive modeling programs available to one of ordinary skill in the art that can be used to model and screen for binding of candidates in silico (see, Hong et al., Evaluation of MHC class I peptide binding prediction servers: Applications for vaccine research, BMC Immunology 20089:8, DOI: 10.1186/1471-2172-9-8, 16 Mar. 2008; Wang, et al., A Systematic Assessment of MHC Class II Peptide Binding Predictions and Evaluation of a Consensus Approach, PLOS, Apr. 4, 2008; Ruppert et al., Prominent role of secondary anchor residues in peptide binding to HLA-A2.1 molecules, Cell, Volume 74, Issue 5, 10 Sep. 1993, Pages 929-937, and also, Nielsen et al., NN-align. An artificial neural network-based alignment algorithm for MHC class II peptide binding prediction, BMC Bioinformatics 200910:296, 18 September 2009, DOI: 10.1186/1471-2105-10-296). Applying these class I and class II models and knowledge to candidate neoantigens will provide useful information that is predictive of the ability of the proposed neoantigen to elicit a CD8+ and or a CD4+ T cell response. This can aid in the fragment selection of the neoantigen and provide a basis for further modification of the structural and chemical properties of the neoantigen.

As good as in silico screens have become, the currently preferred methods of evaluating antigen binding in MHC class I and class II molecules involves an empirical determination of epitope binding, such as with a binding assay. In an exemplary binding assay, a panel of MHC class I and class II molecules representing a variety of haplotypes, is created and screened for each respective molecule's ability to bind candidate antigen peptides. Such panels may be prepared by means described in the art, see for example Justesen et al, Functional recombinant MHC class II molecules and high-throughput peptide-binding assays, Immunome Research, December 2009, 5:2. Binding of antigens to a broad range of common haplotypes is important where the antigen will be prepared for T cell therapies of general use with varying patient genetic backgrounds; whereas for patient-specific approaches the particular physiology of the patient can be targeted. Immunitrack, of Copenhagen, Denmark, is a commercial outsource for MHC class II binding assays currently representing the following alleles: DP: DPA1*0103, DPA1*0202, DPB1*0401, DPB1*0402; DQ: DQA1*0101, DQB1*0301, DQB1*0501; DR: DRA1*0101, DRB1*0101, DRB1*0301, DRB1*0401, DRB1*0701, DRB1*1101, DRB1*1501, DRB3*0101, DRB3*0202, DRB4*0101 and DRB5*0101. EpiVax, Inc. of Providence, R.I. offers DRB1*0101, DRB1*0301, DRB1*0401, DRB1*0701, DRB1*0801, DRB1*1101, DRB1*1301 and DRB1*1501. Currently, the more preferred methods include determinations of immunogenicity, such as T cell proliferation and response assays. T cell assays suitable for measuring the immunogenicity of antigens include: ELISA measuring levels of various activation cytokines, and ELISpot to quantify the frequency of cytokine-producing cells. Flow cytometry permits measuring numerous markers of activated T cells, as well as characterizing relative proportions of T cell subsets in the populations. Nucleic acid based assays for expression of activation markers, and cell proliferation assays are useful, and widely described. PBMCs derived from patients can be screened by T cell assays for a memory response, which can be epitope mapped using epitope-specific peptides. Thus, whether an antigen is subdominant and whether it maintains this status, can be assessed from the patient over the course of treatment.

Cell Populations for Ex Vivo Selection/Expansion of T Cells

To create a reactive T cell population, a source of T cells is needed. Peripheral blood mononuclear cells (PBMCs) are currently preferred, with tumor infiltrating lymphocytes (TILs) being an alternative source. In specific embodiments, T cells may be obtained from the bone marrow, lymph nodes or from other tissue sources.

Circulating lymphocytes obtained from a patient's peripheral blood as well as organ-specific or tissue specific lymphocytes obtained from surgical explants are rich sources of cell populations for ex vivo expansion and preparation for adoptive immunotherapy. T cells present at the site of the disease in the patient are reactive to the disease related antigens. However, they are also subject to an immunosuppressive environment at the site of the disease, such as an inflammation or a tumor, as a consequence of the natural progression of the disease. In case of cancer and tumor related conditions, tumor infiltrating lymphocytes or TILs are isolated, which are tumor antigen experienced, typically a dominant antigen. Tumor reactive T cells are likely to exhibit anergy.

Peripheral blood is the source of circulating T cells, at least a fraction of which have experienced tumor antigens and are therefore primed, or activated. Each T cell can respond to only one antigenic form, characterized by the T cell receptor (TCR) it expresses. These primed cells which exhibit TCR specificity to a particular antigen, when further exposed to the cognate antigen, respond by exponential growth, high level of expression of certain cell surface activation markers, and are positive for antigen specific pentamer binding assays. Identification of dominant and subdominant antigens in a subject is performed by growing the cells ex vivo in presence of the antigens, where the growth and activation of T cells in response to a dominant antigen is likely to outcompete the ones responsive to a subdominant antigen.

In some cases of immunotherapy, such as for autoimmune or inflammatory disease, disease remediation requires suppression of the immune response. Cells of the immunoregulatory phenotypes, such as Tregs may be isolated and selected from the site of the disease, or from circulating blood or from other relevant tissue and suitably expanded using the method described. Suitable cell surface markers for include selection markers comprising CD4+, CTLA-4, CD39, CD73 and CD25+. An isolated cell population is sorted for the given markers to generate enriched T regulatory cells, which are then expanded.

Alternatively, Tregs may be selected out from a population of cells using standard techniques in order to transfer highly reactive T cells, such as in conditions required to augment an efficient immune response.

Circulating T cells express CD45RO, representing the memory phenotype, CD45RA, which exhibit naïve phenotype, CD56, CD57 representing NKT phenotype, CD27 and CD28, representing naïve central memory phenotype, or other surface proteins such as chemokine receptors, tissue homing receptors and activation markers. In case of metastatic diseases, circulating lymphocytes are the source of metastasis antigen reactive T cells, unlike tumor infiltrating cells (TILs), which are reactive only to the tumor antigens in the tumor of origin.

Both antigen naïve and antigen cognate T cells are present in a human peripheral blood, making up about 0.7 to 4 percent of the cellular components in normal subjects. Using optimized cell biology techniques of the present invention, it has been possible to direct a mixed population of cells isolated from the peripheral blood and generate specific subtypes of T cells for cell mediated therapy, where the cells are reactive to a directed set of subdominant antigens and neoantigens.

T cells are also present at the disease site, such as inflammation, an autoimmune reaction, a tumor or an infection, namely a viral, bacterial, fungal, an adventitious or a latent infection. Autologous T cells are obtained from a patient's tissue sample for expansion in vitro by an optimized cell culture method of the invention to obtain a cell population for the immunoreactive therapy. In specific embodiments, T cells may be obtained from bone marrow derived cells from another human subject.

T cells present at the site of the disease are essentially cognizant and reactive to the disease related antigens, but, a vast majority of these T cells are responsive to dominant antigens. In turn, they can be part of an immunosuppressive response, which may be conducive to the disease progression. Such T cells are likely to exhibit anergy.

Peripheral blood is the source of circulating T cells, some of which have encountered disease antigens, such as tumor antigens and are therefore primed. Circulating T cells may also express CD45RO, representing the memory phenotype, CD45RA, which exhibit naïve phenotype, CD56, CD57 representing NKT phenotype, CD27 and CD28, representing naïve central memory phenotype. Additionally, circulating lymphocytes are source of metastasis antigen reactive T cells, unlike tumor infiltrating cells (TILs), which are reactive only to tumor antigens. In specific embodiments, T cells may be obtained from the bone marrow, lymph nodes or from other tissue sources.

Analysis of Initial Cell Population/Culture Setup

Cells are obtained, either previously frozen or freshly isolated, from either healthy donor's blood or from a subject having a medical disorder such as cancer, infection or an autoimmune disorder. Peripheral blood mononuclear cells are obtained from the subject (donor or patient) by standard methods. In certain embodiments, the PBMCs are frozen for later use in the methods of the invention after thawing. In a patient with a disorder, the PBMCs may have experienced antigens related to the disorder. Cells may be seeded in G-Rex10 (Wilson Wolf Manufacturing) gas permeable devices, or grown in any suitable container or device, as deemed feasible by one of ordinary skill in the art.

By way of example using blood derived cells, PBMCs are suspended in cell culture medium. In certain examples presented here, 30-100 million PBMCs are suspended in complete medium. A useful formulation is CellGenix CellGro Medium (CellGenix GmbH), supplemented with 10% Human type-AB serum (Corning Inc.) and 1% GlutaMAX-1 (ThermoFisher Scientific) at the concentration of approximately 2-3 million cells per milliliter. Cells are washed with CTL Anti-Aggregate Wash Medium (Cellular Technology Limited) (CTL-AA-005), and resuspended in CellGenix CellGro Medium. Typically, cell culture procedures are performed using standard temperature and humidity conditions (37 degrees at 5% CO2).

A portion of the cells from the initial population are analyzed by flow cytometry using antibodies (or other suitable methods) for the following markers to check for viable T cells, B cells, Monocytes, and NK cells in the starting population: Live/Dead stain, CD3, CD4, CD8, CD14, CD16, CD19, CD56.

T-Cell Stimulation and Expansion

In the methods described herein, the cell population in culture is exposed to antigens and is treated with one or more cytokine during continuous culture. In one embodiment, stimulation (including priming) of the T cells in the cell population can be performed by exposing the cell population to a peptide mixture derived from one or more target antigens. In embodiments of the invention, the cells are sequentially stimulated with individual target antigens (or polypeptides derived from the target antigen). In other embodiments, the cells are stimulated with multiple target antigens (or polypeptides derived from multiple target antigens) simultaneously.

In embodiments of the invention, the cell population is divided into two or more sub-populations which are each exposed to a peptide mixture derived from a different target antigen. The stimulation is performed as three separate cultures that are pooled prior to final harvest (split pool protocol) or sorted with a GMP compatible FACS instrument like the Miltenyi Tyto. In embodiments, cells can be expanded in culture against each antigen separately and pooled prior to patient administration. Alternatively, cells may be pooled then contacted sequentially with alternative antigen preparations.

Cells may be optionally split following an initial growth phase, which occurs in presence a mixture of antigenic peptides. In the next phase, subpopulations, each responsive to single antigens are grown separately and in the presence of the dedicated antigen to facilitate equivalent representation of a variety of antigens responsive cells. It is possible that certain antigen responsive cells are likely to be lost in the competition for costimulatory molecules or effect on cell growth. Cells grown in the presence of single antigen have different growth requirements and statistics. In case of EBNA1 antigens, for example, individual antigen stimulation results in higher cell yield at day 21, compared to pooled peptide mixes. The cells from split culture are eventually pooled together for a composition of diverse antigen specific cells. Split or pooled cell population undergo the same quality control tests for the release criteria for immunotherapy.

Stimulation of T cells can be accomplished using a variety of methods. Polypeptide antigens are useful to load MHC structures on antigen presenting cells (as discussed above) in the cell population (e.g., PBMCs or a cell population derived from PBMCs). When using purified T cells, antigen-loaded APC populations can be added to the T cell cultures. In embodiments of the invention, peptide antigens are MHC class I or class II optimized.

Polypeptides are commonly suspended in saline, or dimethyl sulfoxide (DMSO), which may be further diluted to required concentrations before adding to the cell culture media. Antigen concentrations will vary depending on the priming technique and toxicity of the antigen, but generally range from 1 nanogram to 10 micrograms of polypeptide per ml of culture medium.

Use of pools of polypeptides derived from one or more target antigens to stimulate the cells ex vivo results in a heterospecific T cell population enriched for T cells that recognize the target antigen or antigens. Such a heterospecific T cell population is generated to trigger a highly active effector cytotoxic T lymphocyte (CTL) response against multiple antigens.

Cytokines, Supplements, and Expanding Heterogeneous Population of T Cells

Stimulation and expansion of T cells in cell culture is supported by a combination of cytokines, such as IL-2, IL-7, IL-12, IL-15 and IL-21, to obtain a proportional increase of heterospecific T cells and to transform them towards specific functional subtypes. In a preferred embodiment, IL-7 and IL-15 are used to stimulate and expand T cells in the methods of the invention. In another preferred embodiment, IL-2, IL-15 and IL-21 are used to stimulate and expand T cells in the methods of the invention.

Each cytokine, alone or in combination, results in certain outcomes in the phenotypic characteristics of the cell population described in Table 2. The culture may be subjected to one cytokine or set of cytokines for a certain period of time, and then the composition is altered to suit the progress of the culture procedure. The following table illustrates uses of cytokine combinations for the specific expansion of the lymphocytes in culture, based on which the cells culture may be subjected to timed exposure to one or more cytokines at the given concentrations.

| Cytokine | Concentration | Outcome |
|---|---|---|
| IL-2 | 10-1000 IU/ml | Terminal differentiation of T |
| | | effector cells; Maintenance |
| | | of Tregs |
| IL-7 | 5-100 ng/ml | Homeostatic proliferation of |
| | | naïve and memory CD4+ |
| | | and CD8+ T cells; |
| IL-15 | 5-100 ng/ml | Expansion of CD8+ memory |
| | | T cells and NK cells |
| IL-21 | 5-100 ng/ml | Increase TCR repertoire |
| | | diversity when used during |
| | | initial stimulation |
| IL-7, IL-15 | 5-100 ng/ml each | Expansion of central |
| | | memory and stem cell |
| | | memory T cells |
| IL-2, IL-7, IL-15 | IL-2 is approximately 1000 | Increase of CD4+ and CD8+ |
| | IU/ml; others 5-100 ng/ml | T cells; Expansion of |
| | each | effector, central and stem |
| | | cell memory T cells |
| IL-2, IL-15, IL-21 | IL-2 is approximately 1000 | Increase in TCR repertoire |
| | IU/ml; others 5-100 ng/ml | diversity, CD8+ memory T |
| | each | cells, stem cell memory T |
| | | cells and NK cells |
| IL-2, IL-7, IL-15 and IL-21 | IL-2 is approximately 1000 | Increase of CD4+ and CD8+ |
| | IU/ml; others 5-100 ng/ml | T cells; Expansion of |
| | each | effector, central and stem |
| | | cell memory T cells; |
| | | Diversify TCR repertoire |

One of the specific advantages of the use of IL-7 in T cell culture is that it promotes antigen specific CD4+ T cell expansion, and is shown to preserve lymphocyte viability and CD62L marker expression in mice, see Ceserta, S. et al., 2010, Eur. J. Immunol., 40: 470-479; Montes, M. et al., 2005, Clin. Exp. Immunol., 142: 292-302. Rosenthal et al., reported that the CD8+ T cell proliferation in response to specific CMV antigens was the highest in presence of IL-15, compared to IL-7 or IL-2. US 2014/356398 discloses that IL-15 rescued CD8+ T cells with a central memory phenotype from death, while IL-7 did not.

Analysis of the Cell Population and Selection of Antigen Activated T cells

Throughout the process the ex vivo expansion and modification of the cells, cells are periodically removed and sampled for quality control examination by analysis of cell surface markers and conditionally subjected to variations in the protocol for achieving the best combination of cells for obtaining the heterogeneous cell population for immunotherapy.

In embodiments of the invention, the T cells in the cell population are selected to enrich for T cells that recognize one or more target antigens. In one aspect, the invention provides a method for isolating T-cells already stimulated by (i) exposure to antigens in the body, or (ii) use of the T-cell stimulation methods described herein. In certain embodiments, the cells are sorted in a closed system sorter. Cells are sorted on the bases of one or more of activation markers, and cell viability. Markers used for determining antigen exposed cell activation profile include CD3, CD4, CD8, CD137(4-1BB), CD297 (PD-1), CD25, CD45RO, CD45RA, CD197(CCR7), CD62L.

An embodiment includes screening of the cells for PD-1 expression, selection of the PD-1 positive cells and growing them in cell culture conditions that will allow robust expansion of the cells.

Another embodiment includes screening of cells for the expression of CD137 on the isolated cells in culture for antigen exposure marker, and subjecting the cells bearing CD137 marker to cell culture conditions that will allow robust expansion of the cells. In another embodiment, a multitude of expression markers including CD-137 and PD-1 are used to select the cells for expansion ex vivo. The expression markers for screening the cells that have been antigen-primed in vivo include one or more members selected from the group comprising CD8, CD274, CD62L, CD45RA, CD45RO, CD-27, CD28, CD69, CD107, CCR7, CD4, CD44, CD137 (4-1BB), CD137L (4-1BBL), CD279 (PD-1), CD223 (LAG3), CD134 (OX40), CD278 (ICOS), CD183 (CXCR3), CD127 (IL-7Rα), CD366 (TIM3), CD25 (IL-2Rα), CD80 (B7-1), CD86 (B7-2), VISTA (B7-H5), CD152 (CTLA-4), CD154 (CD40L), CD122 (IL-15Rα), CD360 (IL-21R), CD71 (Transferrin Receptor), CD95 (Fas), CD95L (FasL), CD272 (BTLA), CD226 (DNAM-1), CD126 (IL-6R), Adenosine A2A Receptor (A2AR).

A preferred system for carrying out the method of the invention includes a sorting device supporting high precision and mild conditions on cells, which preserves maximum cell viability during the procedure. The device is preferably automated and maintains a sterile working system for uptake and dispending of the sorted cells directly into the culture vessels.

Certain subsets of T cells in the population are immunosuppressive (e.g., Treg, TH17, anergized T cells), and their presence induces immune tolerance. These T cell subsets can be sorted for a preferred subpopulation to modulate an immune response towards potentiation or suppression. Alternatively, these cells may be sorted and eliminated from an ex vivo expanding T cell population, where the autologous T cells are used to invigorate the immune response in a patient.

One embodiment of the invention includes selecting the antigen pre-exposed, activated cells from the isolated cell population. Enhanced expression of PD-1, also considered a T-cell exhaustion marker, lymphocyte-activation gene 3 (LAG-3; also known as CD223), T cell immunoglobulin and mucin domain 3 (TIM-3) on CD8+ tumor infiltrating T lymphocytes (TILs) from melanoma patients correlate with antigen exposure and activation. However, while approximately 16% of TILs express PD-1, LAG-3, TIM-3, only 0.3%±0.1% PBMCs from melanoma patients are positive for these markers, see Gros, A. et al., 2014, J. Clin. Invest, 124 (5): 2245-2259. Peak PD1 expression was observed in PBMC-derived T cells after ex vivo antigen challenge, which decreases during culture. Another marker for antigen exposure, 4-1BB (CD137), is a costimulatory marker of the TNF receptor family.

In embodiments of the invention, cells are sorted for expression of priming and action markers, including CD25, CD107a, CD154, CD137, CD279, CD3, LAG-3 or TIM-3 or any suitable marker for antigen primed activated cells. Magnetic bead separation and FACS are techniques for cell separation. In embodiments of the invention, sorted cells are returned to the in vitro culture system for antigen-restricted expansion and or polyclonal stimulation. Efficient, high purity sorting of cells generates at least about 50-fold to about 250-fold increase in antigen directed T cell populations prior to harvesting (by, e.g., day 21) with respect to the day of sorting.

In embodiments of the invention a pentamer assay is used to measure antigen specific T cell expansion. Pentamers are recombinant proteins that are made up of a specific MHC allele bound with a specific peptide. This combination can bind directly to T cell receptors of a particular specificity. Pentamers can be biotinylated or labeled in other ways for use in flow cytometry.

Restimulation

In embodiments of the invention, the stimulation step is repeated with the same protocol as the first stimulation. The cultures from the first and second stimulations are pooled to diversify the population.

In certain embodiments, following isolation and stimulation of cells, the cells are further supplemented with complete medium and restimulated. In one embodiment, the cells are restimulated by autologous feeder antigen presenting cells, in the presence of one or more cytokines. A certain methods restimulation utilizes PBMCs having prior exposure to the antigenic pool, and are non-irradiated, added directly to the culture and then removed by sorting or by adhesion of cells to the culture plate. In a variation of the method, antigen stimulated but irradiated PBMCs are used for restimulating the growing T cells. Alternatively, antigen activated dendritic cells are used instead of PBMC for restimulation.

A method of restimulation utilizes PBMCs having prior exposure to the antigenic pool, and are non-irradiated, added directly to the culture and then removed by sorting or by adhesion of cells to the culture plate. In a variation of the method, antigen stimulated but irradiated PBMCs are used for restimulating the growing T cells. Alternatively, antigen activated dendritic cells are used instead of PBMC for restimulation. However, DCs are the preferred method of antigen presentation where the antigen is a non-peptide antigen, specifically a nucleotide antigen, or an RNA antigen. Preferred peptide antigens are MHC class I or class II optimized. Peptides are commonly suspended in saline, or dimethyl sulfoxide (DMSO), which may be further diluted to required concentrations before adding to the cell culture media. Antigen concentrations will vary depending on the priming technique and toxicity of the antigen, but generally range from 1 nanogram to 10 micrograms of peptide per ml of culture medium.

At this stage, T cells exhibit a variety of cell surface activation markers. The surface markers are identified by antibody reaction to the surface proteins or by performing FACS. Throughout the process the ex vivo expansion and modification of the cells, cells are periodically removed and sampled for quality control examination by analysis of cell surface markers and conditionally subjected to variations in the protocol for achieving the best combination of cells for obtaining the heterogeneous cell population for immunotherapy.

Polyclonal Stimulation

In certain embodiments, expansion of T cells that recognize the desired target antigen(s) is facilitated by polyclonal stimulation of the cell population containing T cells. In preferred embodiments, polyclonal stimulation occurs after the T cells have been stimulated to expand by exposure to one or more target antigens and to certain cytokines as described herein. Preferably, polyclonal stimulation is performed at least about two weeks prior to harvesting the cells.

This polyclonal stimulation may be accomplished by any means that causes the non-specific expansion in the number of T cells. In a preferred embodiment, polyclonal stimulation comprises exposing the cell population to tetrameric antibodies that bind CD3, CD28 and CD2. Other non-specific T cell activators can be used for polyclonal stimulation of T cells including but not limited to PHA (phytohemagglutinin) and PMA/Ionomycin.

Harvesting of T Cells

Cells are harvested and analyzed for their viability and expression of suitable cell surface markers, for example, CD279, CD137, CD223, TIM-3 and other activation markers demonstrating functional efficacy, or by functional assays such as cytokine release assay. Preferably, cells are cultured and sorted in closed environmentally sealed (aspetic) systems, such as Tito (Miltenyi Biotech). Harvested cells are either cryopreserved for future use, pooled, or prepared for infusion into a patient for direct use.

T-Cell Compositions for Immunotherapy

In one aspect, the invention provides a population of T cells that can be used in adoptive cell therapy generated by methods of the invention. Adoptive cell therapy (cellular adoptive immunotherapy) is a treatment used to help the immune system fight diseases, such as cancer and infections with certain viruses. T cells are collected, usually from a patient, expanded ex vivo in order to increase the number of T cells that are able to recognize and kill cancer cells or fight infections. These expanded T cells are given back to the patient to help the immune system fight disease.

The T-cell compositions of the invention have properties that are advantages for use in adoptive T-cell therapy, including one or more of the following: greater than a billion CD3+ cells, greater than 70% CD3+ T cells; predominantly CD8+ versus CD4+ T cells; predominantly effector memory T cells with minimal exhaustion; high expression levels of lymphocyte homing and trafficking markers, and high antigen reactivity (higher than previously published academic protocols). The T cell composition made by the methods of the invention provide enhanced homing to tumors, more efficient of target cells, and less exhaustion for a durable response.

In embodiments of the invention the T cell composition comprises greater than 50%. 60% 70%, 80%, or 90% CD3+ T cells as a percentage of total live cells in the composition. In preferred embodiments, the % of CD3+ T cells is greater than 70%.

In embodiments of the invention, the T cell composition is predominantly (greater than 50%) CD8+ versus CD4+ T cells.

In further embodiments of the invention, the T cell composition is predominantly effector memory T cells with minimal exhaustion as measured by flow cytometry for cell surface markers for memory and exhaustion.

In further embodiments of the invention, the T cell composition has high expression levels of lymphocyte homing and trafficking markers as measured by flow cytometry.

In further embodiments of the invention, the T cell composition has high antigen reactivity (higher than previously published academic protocols) as measured by ELISPOT assay.

T cells isolated and expanded ex vivo represent a dynamic population of cells, which is constantly changing in response to the environmental stimulus applied to the culture in the form of growth factors, stimulants, such as antigens, cytokines and chemokines. The population obtained from the human sample is a heterogeneous population of cells. The heterogeneity is evident from cell surface marker expression and antigen recognition. At the completion of the expansion protocol as the ones described in the present application, the cell population will have acquired structural and functional characteristics distinct from the isolated pool, under the guidance of the cell culture procedure. Based on these culture conditions, the resultant cell population is expected to comprise at least 5% of cells responsive to an antigen, to which the cells have been exposed during the ex vivo cell culture. Consequently, the cell population comprises at least 5% of live, activated T cells responding to a first antigen, and at least 5% cells live, activated cells responding to a second antigen, or a third antigen and so on, where, the antigens are not dominant antigens in the patient.

A population of expanded T cells refer to T cells that have been grown in vitro after isolation from a donor's body. These cells are manipulated to undergo considerable transformative steps in vitro, such that the resultant cells could not have been found in vivo under the circumstance prevalent within the patient or by any natural growth or transformative process in vivo. For example, the transformative steps include subjecting the cells isolated from the tissue to a plurality of antigens, which may include subdominant antigens and neoantigens; and adjusting the cell culture conditions such that the cell population develops largely as antigen-restricted CD8+ cytotoxic T cells accompanied by other effector cells. The cytotoxic T cells are also effective in lysing the target cells. A subpopulation of the effector cells further comprise CD8+ memory cells, which confer long term antigen specific memory, and antigen restricted CD4+ T helper cells expanded in vitro. Isolated cells if merely expanded and reintroduced into the body without specializing them ex vivo, might result in the natural immunodominance to take over due to the influence of the tumor microenvironment.

As used herein, a population of “heterogeneous T cells” refers to a plurality of T cells having reactivity towards one or more different antigens. A heterogeneous population may be reactive towards multiple epitopes of a single antigen. Heterogeneous T cells refer to a non-uniform T cell population. Heterogeneous T cells are also expected to encompass a mixture of discrete T cell subpopulation, identifiable by their function, such as cytotoxic and memory T cells. In contrast, heterospecific T cells refers to the antigen-reactivity of that population. A heterogeneous population may be heterospecific as well.

Adoptive Immunotherapy Using Ex Vivo Expanded T Cells

Using the method for ex vivo T cell expansion disclosed in the present application, a seeding of culture of about 30- to 100 million PBMCs, typically may yield approximately 10-100 million effective T cells for immunotherapy after about 21 days of culture. In certain embodiments, an expansion of about 10-100 fold or more in the number of T cells is achieved after 21 days of culture using the method.

Ex vivo expanded cells are tested for appropriate release criteria to be deemed fit for immunotherapy. In essence, (a) an effective cell number required for the adoptive therapy, (b) cell viability, (c) expression of cell surface markers for effective antigen recognition diversity, (d) an effective mix of desired phenotypes, (e) cellular response with respect to cytokine generation and cytotoxicity for the target cells are included in the release criteria. The general therapeutic protocol is followed as disclosed in our related patent application (U.S. Ser. No. 14/122,036, and in PCT/EP2015/053107), with modifications as necessitated by the clinical condition. Upon infusion of the T cells generated by the method, patients undergo immune reprogramming, since the therapy resets the balance of the immune hierarchy from responsiveness to few or one dominant antigen to a cytotoxic response against multiple antigens, rendering effective remediation of the tumor. Targeting multiple antigens facilitates wider coverage of the tumor region, allowing faster and more effective therapy.

After infusion, the patient is re-profiled by assaying tolerance and immune response from time to time to evaluate the effectiveness of the therapy and to modulate the therapy as deemed necessary. Isolation of PBMC or tissue sample that reflects the immune response of the disease may be obtained at frequent interval and examined for antigen response, in particular, potential loss of any antigen responsiveness by pentamer assay. Regression of the tumor is monitored as a primary outcome. The primary evaluation criteria for the therapy is dependent on the pathological condition being addressed.

Treatment of Disease

In certain embodiments, the invention provides using pools of antigens to stimulate and expand a population of cells comprising T cells to generate a composition for immunotherapy or adoptive immune cell therapy comprising T cells with specificity to multiple target antigens. In one embodiment, the invention provides a population of autologous T cells for immunotherapy, wherein the therapy is directed against multiple antigen (e.g., viral antigens, tumor-associated antigens, subdominant antigens and/or neoantigens). These T cell compositions have the ability to provide primary therapy, and efficacious long-term tumor regression without necessitating chemotherapy. Preferentially, such a heterogeneous T cell population is developed to trigger a highly active effector cytotoxic T lymphocyte (CTL) responses against multiple antigen (e.g., viral antigens, tumor-associated antigens, subdominant antigens and/or neoantigens).

In certain embodiments, the compositions and methods of the invention are directed towards, but not limited to, treatment of cancer, solid tumors, blood related disorders, autoimmune, inflammatory and infectious diseases. In certain embodiments, the method is amenable to redirecting any chronic disease, characterized at least in part by immune tolerization or immune suppression, to an acute response against the causal element. Specific examples may include chronic infections such as hepatitis, or latent infections such as tuberculosis, and certain viral infections. By redirecting the suppressed immune response to an active response against multiple subdominant antigens and neoantigens of the pathogen, using variations of the composition and method of the invention, it is possible to ameliorate the disease.

The composition and methods are directed towards but not limited to disease indications such as glioblastoma, non-Hodgkin's lymphoma, gastric, nasopharyngeal, pancreatic, lung and other solid tumors and also hematological cancers. Glioblastoma is particularly important because it is a highly malignant aggressive form of tumor in the brain. It arises from astrocytes, but contains mixed cell types. Because of the presence of different cell types and grades within this tumor it is difficult to treat. Additionally, a complex architecture renders it difficult for surgical excision. Radiation and chemotherapy are used to slow the progress of the disease, with a median survival of about 14.6 months in adults with aggressive glioblastoma (American Brain Tumor Association, http://www.abta.org/brain-tumor-information/types-of-tumors/glioblastoma.html). Due to heterogeneity of this tumor, it is rightly known as Glioblastoma “multiforme”.

Therapeutic attempt of Glioblastoma using EGFRvIII CAR cells resulted in initial success followed by recurrence of the tumor, as 82% of the tumors had lost the EGFRvIII expression, see Johnson L. A. et al., 2015, Sci. Trans. Med, 7(275): 275ra22. Therefore, targeting Glioblastoma at single specific antigen does not provide adequate benefit, and is therefore one exemplary case where the present invention is particularly useful. The need for an improved strategy is visualized in particularly this type of cancer, for which immunotherapy targeting multiple subdominant antigens and neoantigens is likely to be the only effective therapy.

Tumor-edited T cell responses result in T cell fixation on dominant antigens and increased selection pressure against tumor antigens, which mutate in response. U.S. application Ser. No. 14/122,036 (incorporated herein by reference) details reprogramming the immune response by enhancing T cell responses to subdominant antigens, using the cells to therapeutically change cellular homeostasis and the nature of the immune response away from one dominant antigen, towards a different one, in order to break immune tolerance and restore cellular and humoral anti-tumor responses.

By stimulating and growing in tissue culture the T cells from a donor or a patient that recognize particular antigens, and then transplanting them into the patient, the transplanted cells overwhelm the endogenous dominant antigen-restricted T cells and modify the immune response toward the new antigen provided sufficient numbers of T cells are expanded and transplanted. When memory cells are established, they are then reflective of this new immunodominance hierarchy so that the desired therapeutic effect is long lasting. In effect, the transplantation of exogenously generated T cells reactive to particular antigen(s) (e.g., neoantigens) recapitulates priming and rebalancing the patient's immune response to target antigens and produce a therapeutic benefit.

In addition to cancer therapeutics, the principles of immune reprogramming apply to other disease-associated antigens that can be validated by the methods described, such as those associated with chronic and latent infectious agents, for example, agents associated with, viruses, bacteria, fungi, parasites, or prions. Alternatively, with certain antigens that are associated with autoimmunity, neurodegeneration, allergy, inflammation or organ transplantation rejection or graft vs. host disease, it is desirable to induce long-term tolerance. Therefore, certain antigens can be validated as inducing tolerance or down-regulation of Th1 and Th2 responses, inflammatory cytokines, NK cell responses, and the complement pathway

Besides cancer, the method of immunotherapy described herein, comprising redirecting the patient's immunodominance hierarchy to target multiple under-represented or non-represented antigens in order to mount an effective immune response is highly adaptable in various other immunological diseases. It is particularly useful in transforming chronic conditions and immune-subversive infections, such as chronic hepatitis infections; and latent infections such as tuberculosis; as well as different kinds of viral infections, into an effective acute immune phenotype. Likewise, the described method of immunotherapy is amenable to treat disease conditions marked by either a skewed immune response, or else hyperactive allergic immune response, and conditions related to other chronic ailments, including but not limited to autoimmunity.

It is to be understood and expected that variations in the principles of invention herein disclosed can be made by one skilled in the art and it is intended that such modifications are to be included within the scope of the present invention. The following Examples further illustrate the invention, but should not be construed to limit the scope of the invention in any way. All references cited herein are hereby incorporated by reference in their entirety.

### EXAMPLES

### Example 1. T Cell Expansion Platform

Experimental Procedures

Antigen selection: PepMixes are a pool of peptides (also referred to herein as “polypeptides”) derived from a peptide scan of the target antigen of interest (each polypeptide is 15 amino acids with 11 amino acid overlap) that are capable of stimulating CD4+ and CD8+ T cells without the requirement of knowing HLA restriction. PepMixes for LMP1, LMP2, EBNA1, CMV, NYESO-1, and Survivin were purchased from JPT Peptide Technologies, Berlin. Each vial of pepmix consisted of approximately 15 nmol or 25 micrograms of each peptide at 70% purity. Individual LMP2 peptides and custom epitope mapping matrix pools were also purchased from JPT Peptide Technologies, Berlin.

Listed are the Pepmix compositions and source of the protein sequence of the antigens used to expand T cells from normal donor and cancer patients:
PepMix EBV(LMP1): Pool of 94 peptides derived from Latent membrane protein 1, Swiss-Prot ID: P03230 of Epstein-Barr virus (HHV4).
PepMix EBV(LMP2): Pool of 122 peptides derived from Latent membrane protein 2, Swiss-Prot ID: P13285 of Epstein-Barr virus (HHV4).
PepMix EBV(EBNA1): Pool of 158 peptides derived from Epstein-Barr nuclear antigen 1, Swiss-Prot ID: P03211 of Epstein-Barr virus (HHV4).
PepMix HCMVA(pp65): Pool of 138 peptides derived from the 65 kDa phosphoprotein, Swiss-Prot ID: P06725 of Human cytomegalovirus (HHV-5).

Source of PBMCs: Frozen PBMCs from normal healthy donors and cancer patients were purchased from commercial vendors or otherwise isolated from purchased units of whole blood, processed in house, then cryopreserved and stored in the vapor phase of a liquid nitrogen storage vessel.

PBMC Isolation: Peripheral blood mononuclear cells (PBMCs) were prepared by centrifugation over Ficoll-Hypaque gradients. Cells were harvested, washed and resuspended in CryoStor 10 freezing media (BioLife Solutions) in aliquots of 50 million viable cells per vial. Vials were frozen using either a programmable rate controlled freezer (Thermo Fisher) or passive freezer (Nalgene, Mr. Frosty) then transferred to the vapor phase of a liquid nitrogen storage vessel and the locations recorded. Surface immunophenotyping of frozen-thawed PBMCs were performed by flow cytometry to determine the distribution of monocytes, T cells, B cells, and NK cells in starting material.

Donor PBMC screening for EBV reactive T cells: Enzyme-linked immunospot (ELISPOT) kits were used to determine the frequency of T cells secreting interferon gamma (IFN-γ) in response to EBV LMP1, LMP2, and EBNA1 pepmixes, matrix pepmixes, and individual peptides (JPT Peptide Technologies, Berlin, Germany). Cells were plated at 400,000 to 600,000 cells per 96 well, cultured for 18-24 hours, and processed according to the manufacturer's ELISPOT kit protocol (CTL, Shaker Heights, Ohio) and data graphed with GraphPad Prism software.

Results

EBV was chosen as the model for the T cell expansion platform because the human T cell response to the virus and the genes and proteins expressed during its lytic and latent life cycle have been characterized. The Epstein-Barr virus (EBV) is a gamma-herpes virus which establishes latent, life-long infection in more than 95% of the human adult population. The latency pattern 2 shown in FIG. 5a is expressed in several EBV associated cancers. In nearly all nasopharyngeal carcinomas (latency II), LMP1 and LMP2, as well as EBNA1, are expressed. Furthermore, when screening for PBMCs from cancers associated with EBV, 10-20% of Non Hodgkins Lymphoma, 30-50% of Hodgkin Lymphoma, and 10% of Gastric Carcinomas are EBER+(EBV-encoded RNA) and should express Latency 2 antigens EBNA1, LMP1, and LMP2.

Frozen PBMCs from 16 normal healthy donors were screened by ELISPOT (enzyme-linked immunospot assay) for interferon γ (IFN-γ). Approximately 500,000 unstimulated PBMCs were plated in triplicate with 1 μg/ml of LMP1, LMP2, and EBNA1 pepmixes as well as a DMSO negative and PHA positive control. The number of antigen specific spots were divided by DMSO alone background counts to determine the relative frequency of total T cells that responded to each antigen. Out of 16 normal donors tested, 5 out of 16 responded to LMP1, 10 out of 16 responded to LMP2, and 14 out of 16 responded to EBNA1. PBMCs from 5 of the 16 original donors had T cells that responded to all three antigens and were selected as the source of starting material for setting up and optimizing small scale culture conditions. FIG. 5b.

LMP2 peptide epitope mapping: LMP2 pepmix response was further refined by screening donors that responded to LMP2 matrix peptide mixes that narrowed the response to one peptide. FIGS. 5c and 5d lists the individual peptides that are arranged in the LMP2 matrix pools, the IFNγ ELISPOT response of each normal donor to the Matrix pools (1-23), and the identification via matrix selection of the individual LMP2 peptide and minimal peptide sequence that should bind to specific class I HLA molecules specific to the donor.

LMP2 subdominant epitope mapping: IFNγ ELISPOT from Donor HHU20130423 demonstrated the presence of potential T cell dominant and subdominant peptide epitopes from unstimulated PBMCs. Dominant LMP2 peptide 50 was identified by being shared in matrix pools 6 and 16. Subdominant or lower responses to peptide 112 (Matrix pool 2 and 22) and 69 (Matrix pool 3 and 18) were also identified. This donor was identified as recognizing three different peptide epitopes within the same LMP2 molecule with one dominant peptide and two subdominant peptides responses at the time of blood donation. FIG. 5e.

### Example 2. T Cell Culture Conditions

Experimental Procedures

Cytokines: GMP grade cytokines for use in T cell expansion were purchased from Miltenyi Biotech and stock solutions were prepared at 25 ug/ml in sterile dH20 and stored at −70 degC. Cytokines were used at Human IL-2 100 IU/ml final concentration and 10 ng/ml for IL-7 and IL-15.

Frozen PBMCs were stimulated with 1 ug/ml Pepmix during extended culture or at 3 ug/ml during 2 h pulse followed by pooling. DMSO concentration with 1 ug/ml Pepmix culture was 0.4% and no cellular toxicity was observed. PBMCs pulsed with 3 ug/ml Pepmix had a 1.2% DMSO concentration during incubation which was washed away prior to extended cell culture. Flow cytometry was performed on aliquots from Days 0, 7, 14, 21 and or 28 of T cell expansion. Frozen Day 0 PBMC samples were stained for antibodies to characterize starting cell populations: live/dead, anti-CD3 for total T cells, CD8 and CD4 subsets, CD14/CD4 for monocytes, CD56 for NK cells, and CD19 for B cells. On Day 7, cultures were stained for markers of T cell activation/maturation in addition to live/dead, CD3, CD4, CD8, Pentamer (if available), CD45RO, CD45RA, CD197, CD137, CD25, CD62L, CD297. On Days 14, 21, and or 28, the cells are tested for intracellular cytokine response to pepmixes. The ICS staining cocktail is comprised of live/dead, CD3, CD4, CD45RO, CD45RA, CD62L, CD107a, TNFα, IFNg, and IL-2. Memory T cell markers are evaluated on cultures on either Day 21 or Day 28. The Memory T cell staining cocktail is comprised of live/dead, CD3, CD4, CD8, CD45RO, CD45RA, CD197, CD28, CD122, CD127, CD183, CD95, and CD62L

Intracellular Cytokine Staining: CD107a and cytokines (TNFa, IFNg, IL2) Stimulate 0.1-1 million cells in 100 ul media with 10% Human AB serum, 1% Glutamax, and 2 ug/ml Pepmix or DMSO as control. Add 100 ul media with anti-CD107a and 2 ul/ml GolgiStop. Incubate cells for 5 hours at 37 degC. Spin down to pellet cells, remove supernatant, and stain with surface antibodies. Resuspend cells in 100 ul 2% formaldehyde and leave overnight, 4 degC (covered in foil). The next day, wash cells 2-3 times with Intracellular Staining Perm Wash Buffer (Biolegend). Stain with desired intracellular antibodies to cytokines diluted in Perm Wash Buffer, incubate 30 min, 4 degC, in the dark. Wash 2-3× in Perm Wash Buffer. Resuspend cells in 100 ul 2% formaldehyde prior to running samples on flow cytometer or storing in the dark at 4 degC.

Results:

Evaluation of PBMCs from 6 normal donors (FIG. 6a) and another 2 normal donors (FIG. 6b) that were cultured at small scale with LMP1, LMP2, EBNA1 pepmixes did not demonstrate substantial advantage between the three cytokine cocktails (KI: 1000 IU/ml IL-2, 10 ng/ml IL-15/IL-21; 10 ng/ml IL7/15; 10 ng/ml IL15 alone) (FIG. 6a). The second cytokine evaluation study (FIG. 6b) also evaluated the difference in T cell response if all three pepmixes comprising (374 peptides total) vs individual pepmixes (LMP1 94 peptides; LMP2 122 peptides; EBNA1 158 peptides). The arrows in FIG. 6b demonstrate that using all three pepmixes together for stimulation for both PBMC donors inhibits the response to EBNA1. The response to LMP2 is lower for both donors but the drop in activity is not as severe as that seen for the EBNA1 reactivity. The epitope(s) that stimulate EBNA1 are susceptible to competition with peptides in the LMP1 and or LMP2 pepmix. This result led to a modification of the T cell expansion protocol where pepmixes are used individually for pulsing then PBMCs, peptides removed, and PBMCs combined for each antigenic stimulation.

FIGS. 6c and 6d show the result of Donor 109 PBMCs stimulated only with the LMP2 pepmix. At Day 11, 79.0% of the T cell culture was recognized by the pentamer B40:01-IEDPPFNSL and similar antigen specific reactivity was detected by increases of CD107a, TNFa, and IFNg expression. In addition to the high antigen reactivity, FIG. 6d. shows that the Donor 109 CD8+ T cells stimulated with LMP2 pepmix converts phenotype from CD45RA naïve cells to CD45RO Effectory Memory cells. CD62L, another memory marker, as well as activation markers CD25 and CD137 are clearly upregulated between Day 7-11 of culture. FIGS. 6e and 6f demonstrate the overall response for Donors 423 and 915 when stimulated with pepmix individually. Thus, PBMCs may be stimulated individually at process scale then pooled prior to harvest. However, this method triples the size of the batch and increases workload and cost. The “pulse” then “pool” method for stimulating PBMCs with multiple pepmixes was chosen for further development.

### Example 3. T Direct Small Scale Expansion of Non-Hodgkins Lymphoma Clinical Samples

Experimental Procedures:

Flow cytometry: 200,000 cells were stained with antibody panels following standard flow cytometry procedures.

PBMC panel: live/dead, CD3, CD4, CD8, CD14, CD56, CD19.

Intracellular cytokine expression: live/dead, CD3, CD4, CD8, CD45RO, CD45RA, CD107a, TNFa, IFNg, IL-2.

T cell activation panel: Antigen specific pentamer, live/dead stain, CD3, CD4, CD8, CD56, CD45RA, CD45RO, CD25, CD62L, CD137, CD197, and CD279.

T cell Memory panel: live/dead, CD3, CD4, CD8, CD45RO, CD45RA, CD197, CD28, CD122, CD127, CD183, CD95, and CD62L.

Small scale expansion protocol: On Day 0, 2 vials of NHL frozen PBMCs (HemaCare, Donor NHL 14103815) were thawed using CTL anti-aggregate solution according to manufacturer's protocol. Cells were washed and resuspended in CellGro DC Media (CellGenix)+10% Human AB Serum (Corning)+1% GlutaMax (Gibco), and an aliquot was removed for counting and FACS analysis with PBMC and T cell activation panels. Approximately 2 million PBMCs were pulsed with 3 μg/mL LMP1, LMP2, or EBNA1 Pepmix for 2 hours at 37 C. After the incubation time, cells are washed, resuspended then pooled for a total volume of 2 ml and transferred to one well of a GREX 24 well plate (Wilson Wolf). Cytokines were added to either a final concentration of 10 ng/ml IL-7/IL-15 or 10001 U/ml IL-2, 10 ng/ml IL-15/21 (KI cytokines) for a 28 day culture. On Day 7, cells were resuspended, counted, and stained for activation markers with T cell panel (live/dead, CD3, CD4, CD8, Pentamer (if available), CD45RO, CD45RA, CD197, CD137, CD25, CD62L, CD297). The culture was restimulated by repeating the Day 0 stimulation protocol with new frozen PBMCs and then combined with the Day 7 culture (4 ml total). Cultures were fed every 2-3 days with fresh media containing cytokines.

On Day 14 the cultures were analyzed by intracellular cytokine staining by restimulating an aliquot of each culture with (i) DMSO, (ii) LMP1 Pepmix, LMP2 Pepmix, or (iv) EBNA1 Pepmix. The remaining Day 14 culture is transferred (˜6 mL) from the 24 well plate into a GRex-10 or GRex 6 well plate. Cell cultures were fed with 5 ml of cytokine media containing IL-7 and IL-15, and then 250 μL (25 μl/ml) of ImmunoCult CD3/CD28/CD2 human T cell activator (Stemcell Technologies) was added. Cell cultures were fed with cytokine media on Days 15 and 18. Samples were tested again on Day 21 for intracellular cytokine expression. Day 28 was the final day of culture and a sample was tested by flow cytometry for the T cell activation panel, T cell Memory panel, and intracellular cytokine expression. Remaining sample was harvested and cryopreserved.

Results:

NHL sample HemaCare, Donor NHL 14103815 was cultured with two different cytokine cocktails (10 ng/ml IL-7/IL-15 or 1000 IU/ml IL-2, 10 ng/ml IL-15/21 (KI cytokines)) with or without polyclonal T cell activator ImmunoCult CD3/CD28/CD2 for a total of 28 days. Evaluating the condition of the cells at Day 28 harvest, the IL7/15 cytokine cocktail combined with CD3/CD28/CD2 polyclonal stimulation generated cells with the best profile: >97% CD3+(FIG. 7a), increased CD107a expression in response to stimulation (LMP1: 10.7%, LMP2: 1.42%, EBNA1: 0.77%, DMSO: 0.49%). FIG. 7b. No clear growth advantage was observed from either IL-7/15 vs KI cytokines and addition of CD3/CD28/CD2 polyclonal stimulation. However, the additional benefit of reintroducing CD3/CD28/CD2 polyclonal stim was seen in day 28 memory staining, where the percentage of potential memory cells was increased in the presence of CD3/CD28/CD2-as measured by expression of CD197 and other markers. FIG. 7c.

Another NHL sample from a patient with Stage 1 Follicular Lymphoma was cultured with the same IL-7/15 and ImmunoCult CD3/CD28/CD2 polyclonal stimulation for a total of 28 days. FIG. 7d. demonstrated that this protocol successfully expanded LMP1 (7.91%), LMP2 (26.0%), and EBNA1(5.09) specific T cells (DMSO control 0.91%). T cells specific for subdominant latent antigens LMP1, LMP2, and EBNA1 can be expanded directly from PBMCs without the need for stimulation with dendritic cells. The advantage of this process is that dendritic cells and viruses are not required to maximally stimulate T cells and should be linearly scalable for manufacturing (>1 billion) large number of T cells.

### Example 4. T Direct Process Scale Expansion of Normal Donor PBMCs

Experimental Procedures:

T Direct Production Scale protocol (yield of >2 billion cells): Pepmixes were dissolved in 100 μl of CryoMACS GMP grade DMSO (Miltenyi Biotec) until completely dissolved (visual inspection). Cryopreserved PBMCs were thawed using CTL anti-aggregate solution and washed twice with serum free RPMI-1640. Cells were resuspended at 10 million/ml in production media (CellGenix GMP DC Medium, 10% Access Biologicals Human AB Serum, 1% Glutamax). 6-10 million PBMCs were stimulated with respective pepmix at 1-5 ug/ml in production media for 2 hr at 37° C. After incubation, the cells were washed, each pepmix stimulated culture resuspended in fresh production media, then combined for a total volume of 15 ml, and transferred to a GREX10 culture vessel. IL-7 and IL-15 cytokines were added to a final concentration of 10 ng/ml.

After 7 days in culture, cells were counted and immunophenotyped by flow cytometry using a T cell activation panel (Antigen specific pentamer, live/dead stain, CD3, CD4, CD8, CD56, CD45RA, CD45RO, CD25, CD62L, CD137, CD197, and CD279). Cells were gated for live/dead, CD3+, and the percentage of CD137+CD25+ T cells evaluated as a surrogate marker for antigen response. Day 0 stimulation protocol was repeated with the same donor PBMCs (15 ml) and added to the day 7 culture for a final volume of 30 ml in production media supplemented with 10 ng/ml IL-7 and IL-15. Media was changed every 2-3 days based upon visual inspection of culture.

Hydrophobic peptide sequences often aggregate to form crystals and should be removed prior to Day 14 either by centrifugation of cell culture on Ficoll-Hypaque gradient or cell filtration filters. Alternatively, pepmixes were diluted to appropriate concentration in production media and passed through a 0.22 micron sterile filter to remove the majority of insoluble peptide crystals.

On Day 14, the culture was tested for antigen specific reactivity by intracellular cytokine staining for cell surface activation markers, CD107a, TNFa, IFNg, and IL-2. Cells were resuspended to a concentration of 1 million cells/ml (typically 100 million cells at this stage), transferred to a GREX100M (1 liter capacity) and stimulated with a CD3/CD28/CD2 Immunocult humanT cell Activator (StemCell Technologies). Fresh production media with 10 ng/ml IL-7 and IL-15 was added every 2-3 days. Cells were harvested on Day 28 and release testing performed for % CD3 cells (>70%) and sterility. Harvested material was resuspended in CryoStor 10 and frozen in 50 ml bags (Miltenyi Biotec) or cryogenic vials (Corning).

Cytotoxicity Assay: LDH Cytotoxicity Detection Kit. The cytotoxic T lymphocytes (CTLs) were tested for specific cytotoxicity against autologous T cell blasts pulsed with either DMSO or specific pepmixes during the last 24 hours of PHA culture. Cell-mediated cytotoxicity of antigen-specific T cell effector cells was measured with the LDH Cytotoxicity Detection Kit (Takara, Cat # MK401). Autologous PBMCs were stimulated with PHA to generate T cell blasts. T cell blasts were pulsed overnight with DMSO or specific pepmixes, harvested, dead cells removed (ClioCell Magnetic Beads), and plated at 10,000 cells per well in serum free media. Effectors from either day 21 or day 28 products were harvested, characterized by flow cytometry for antigen reactivity (intracellular cytokine staining) then frozen until targets were ready for the assay. Assays were set up with Effector to target (E:T) ratios that ranged from 5:1 up to 20:1 dependent upon the number of effector cells. Assays were incubated for 6 hours, supernatants harvested, and the increase of LDH enzymatic activity measured using the kit protocol.

Results:

T cell expansion from normal healthy donor PBMCs was performed at what we consider as process scale. FIG. 2 is a schematic outlining important steps for the process. Compared to historical methods for expanding large numbers of EBV-specific T cells, the method described here is straight-forward yet effective. The cell number yield at Day 28 harvest was over 2 billion viable cells with a CD3% of >95%. The product is predominantly CD8+(63%) with 12.5% of the total CD3 population expressing CCR7 and more than half the CD3 cells expressing the chemokine trafficking receptor CXCR3. FIG. 8a. After 28 days, the T cells upregulated CD107a, and produced TNFα, IFNg, and IL2 in response to all three pepmix antigens (FIG. 8b). FIG. 8c demonstrates dose dependent selective killing of targets (T cell blasts loaded with LMP2 or EBNA1 pepmixes) by donor 109 T cell expansion product at 20:1, 10:1, and 5:1 effector to target ratios using a non-radioactive cytoxicity assay that measures LDH from damaged cells.

### Example 5. T Select Process

Experimental Procedures:

T Select Process involves sterile cell sorting of low abundance T cells either from T expansion cultures stimulated with known antigens—viral proteins, overexpressed cellular proteins, mutated cellular proteins, peptides. The method of stimulating T cells is identical to the expansion process provided in Example 3 except that cells are sorted for activation (CD 137 and CD25) between Day 7 and 11, and then returned into culture with media containing cytokines. If the antigen reactivity (determined by intracellular cytokine response to antigen) is still below 5% after cell sorting and culture, then the CD3/CD28/CD2 Immunocult humanT cell Activator reagent is used.

Activated PBMCs were stimulated with selected pepmixes and the level of CD137+CD25+CD8 T cells were characterized then sorted on the MacsQuant Tyto Sorter (Miltenyi Biotec), placed back into culture with IL7/15 containing media. Cells were used as effectors for killing of autologous T cell blasts loaded with specific pepmixes by the procedure described in Example 4.

Results:

Gros et al. reported the capture of rare populations of cancer neoantigen specific T cells directly from the blood of melanoma patients (Gros et al. Nature Medicine:22, 433-438, 2016). The expression of PD-1 identified a diverse and patient-specific antitumor T cell response in peripheral blood. In addition to PD-1, other markers of activated or exhausted T cells could be used for isolating antigen specific cells after 7 days in culture (see FIG. 9e). Day 7 expanded PBMCs were evaluated for expression of T cells expressing CD137 and CD25 (FIG. 9d). CD3+CD137+CD25+ populations could be used to sort and enriched for antigen reacted T cells that have extremely low precursor frequencies. Furthermore, the isolation by cell sorting of activated markers could be applied to improvement of T Direct when the percentage of activated T cells is below 5% at Day 7. On Day 7 the cell culture would be stained with a T cell activation panel including individual or combinations of the following antibodies: CD69, CD279(PD-1), CD223(LAG3), CD134(OX40), CD183(CXCR3), CD27(IL-7Ra), CD137(4-1BB), CD366(TIM3), CD25(IL-2Ra), CD80, CD152(CTLA-4), CD28, CD278(IOS), CD154(CD40L), CD45RO.

Donor 109 T cells were evaluated for CD137 expression and LMP2 specific pentamer staining at Day 6 and Day8. The percentage of pentamer positive CD8+ Tcells is similar to cells gated for CD137+CD25+. CD137+CD25+ markers designate an antigen activated T cell population and can be used for isolation of antigen specific T cells, either from T cell cultures or directly from patient blood. Donor 109 Day 7 cultures were sorted on the Tyto (Miltenyi Biotec) and the material demonstrated >90% purity, good viability, recovery, and morphology post sort (FIGS. 9f and 9g). Sorted cells were expanded in media containing IL7/15 cytokines and demonstrated selective cytotoxicity against peptide loaded T cell blasts as targets (FIG. 9h).

Stage IV Glioblastoma and Pancreatic Cancer PBMCs: PBMCs were cultured at small scale using KI cytokine cocktail (100 IU/ml IL-2, 10 ng/ml IL15/IL21) and individual pepmixes for CMVpp65, NYESO-1, and Survivin. The presence of antigen activated T cells was evaluated by detection of CD137+CD25+CD8+ T cells using flow cytometry. CMVpp65 specific T cells predominate in both donors. GBM Day 14 cultures were analyzed by intracellular cytokine staining for TNFα production and response to cellular tumor antigen NYESO-1 and Survivin was only 3.6 fold over background. The NYESO-1 and Survivin populations were 7-9 fold over background for the pancreatic cancer Day 14 culture.

### Example 6. Identification and Selection of Neoantigens for Use in T Cell Selection and Expansion Protocols

The following example describes selection of neoantigens for use in generating antigen-restricted T cell populations, that are reactive against glioblastoma and other cancers. The example details expression analysis of the tumor associated antigens for immunotherapies targeting glioblastoma, and the use of genomics and tumor evolution to select neoantigen specific peptides. We exemplify herein both personal neoantigens (specific for each patient) and shared neoantigens (i.e. those genes which are mutated in tumors from more than one patient, and in more than one tumor type). As such, validating tumor associated antigens in glioblastoma using genomics/tumor evolution/bioinformatics is exemplary and we use the same approach in other cancers.

These mutations are point mutations or recombinations at mutational hotspots in expressed proteins specific to only the tumor and preferably those that are shared in primary and recurrence (local and/or metastatic) and more preferably in all/most cancer cells in the tumor. These peptides selected by genomics approaches may need to be picked individually and tested further for binding (using net MHC or MHC binding and/or T cell assays). Most preferably one wants to demonstrate that the neoantigens used only expand T cells reactive with the mutant but not the normal (wild-type) protein in the target patient.

The neoantigens herein provide for panels of candidate antigens representing types of tumors (e.g.—gliomas or glioblastomas) and even pan-cancer panels. To the extent these panels can be aligned with the sequencing and identification of these mutations from blood, one can identify the antigens in the blood using sequencing of circulating DNA in the plasma then grow T cells from PBMCs form the same patient's blood.

Certain data for this example comes from The Cancer Genome Atlas (TCGA) Glioblastoma project, published in Cell 2013 Oct. 10; 155(2):462-77 (incorporated herein by reference). That study provided genomic data from 580 patients. Next-Generation sequencing was carried out in 291 samples.

The first step is extracting genes that are recurrently mutated in several patients. Genes with a role in tumors (driver genes) that are mutated more than expected can be found with several tools including MutSig (Broad, Nature 499, 214-218 (2013)), MutComfocal (Columbia U., Nature Genetics 2013). Using standard Mutsig analysis in the above cohort 11 genes were identified (PIK3R1 PTEN TP53 EGFR IDH1 BRAF PIK3CA RB1 NF1 PDGFRA LZTR1). Point mutations in these genes occur in 70% of GBM cases. See FIG. 10 and the Table below.

| | |
|---|---|
| BRAF | v-raf murine sarcoma viral oncogene homolog B1 |
| EGFR | epidermal growth factor receptor (erythroblastic |
| | leukemia viral (v-erb-b) |
| | oncogene homolog, avian) |
| IDH1 | isocitrate dehydrogenase 1 (NADP+), soluble |
| LZTR1 | leucine-zipper-like transcription regulator 1 |
| NF1 | neurofibromin 1 |
| PDGFRA | platelet-derived growth factor receptor, alpha polypeptide |
| PIK3CA | phosphoinositide-3-kinase, catalytic, alpha polypeptide |
| PIK3R1 | phosphoinositide-3-kinase, regulatory subunit 1 (alpha) |
| PTEN | phosphatase and tensin homolog; |
| | phosphatase and tensin homolog |
| | pseudogene 1 |
| RBI | retinoblastoma 1 |
| TP53 | tumor protein p53 |

FIG. 10 shows mutation frequency. Each column is a single patient. The second column is the frequency of the mutation in all GBM patients. For example, first patient has mutations in PIK3R, PTEN, p53 and RB. These mutations are not independent and a patient could have several alterations in these genes. These associations are statistically relevant in some of these genes. As only expressed proteins could result in antigens, this mutational analysis focuses on point mutations that result in expressed proteins. For example, there is an association between P53, IDH1 and ATRX and CDK2a being mutated together. However, because ATRX and CDK2a are mutational deletions, they are not included in our analysis. But the pairs above are expressed point mutations and because of the correlation, both neoantigens could be targeted at once with T Direct or T Select in the same tumor. Fusion proteins could also be a target. One useful fusion protein that presents as a target is EGFR/TAC-3 (NKB). The mutation always happens the same place and is a recombination hot spot that is present in 3 to 5% of Glioblastoma, and appears an early event-driver event (spread across the tumor). Other fusions such as EGFR/CEP14 are highly expressed in 8% of the tumors but are a late event and subclonal thus are not optimal targets.

Next we examine how the mutations in affected patients distribute along the selected genes. Turning to FIG. 11 with regards to BRAF, there is a significant focused hotspot making it a good preliminary neoantigen candidate target. But it presents in a small fraction of glioma patients (<2% of adult gliomas). When present however, it provides a highly conserved target. Likewise, a very large fraction (40-50%) of melanomas display the BRAF mutation, and leukemias (e.g., 40% Hairy Cell Leukemia have BRAF mutations. This mutation is also present in colorectal cancer (10% BRAF mutations), lung and papillary thyroid cancer, certain brain tumors have it (10-15% pilocytic astrocytoma, 5-10% of pediatric diffusely infiltrating gliomas, anaplastic astrocytomas and glioblastomas and between 30% to 60% of gangliogliomas).

FIG. 11 also shows the neoantigen candidate EGFR. Mutations scattered with the 289 hotspot render this a useful target. FIG. 11 also shows that neoantigen IDH1 is an optimal target for our T cell therapy. IDH1 R132G/H is always the same (a highly conserved hotspot) and is a founder event-thus found in primary and recurrence and early in the trunk-all the branches. This mutation is found in 5-10% of Glioblastoma and 70% of Low Grade Gliomas. It is also found in AML, Peripheral T cell lymphomas and acute PML and some myelodysplasias (MDS) sometimes associated with transition to premalignancy. IDH1 mutations in Low Grade Gliomas and Glioblastoma correlate with better prognosis. FIG. 11 also shows LZTR1. This is a useful neoantigen where the mutations targeted represent multiple regions of the peptide and overlapping or contiguous fragments are used to prime T cells. Conversely, for neoantigen NFI, the majority of mutations make for truncated proteins, and so are not useful antigens for generating a T cell response. See FIG. 11. Neoantigen PDGFRA has mutations throughout. See FIG. 11. E229K is a useful target and is present in about 2% of patients. As shown in the corresponding panel in FIG. 11, PIK3CA is a good neoantigen to target. E545 A/K is a hot spot which is present in 5% of glioma and glioblastoma patients. FIG. 11 also shows PIK3R1 is a good neoantigen target based on the G376R hotspot, which is present in about 4% of patients. FIG. 11 also shows the neoantigen PTEN. PTEN has a high frequency of mutations due to its length, but it displays many inactivating mutations that create stop codons. Therefore, while the antigen common and correlates with other neoantigen genes which are also mutated, it is not as useful to create T cell responses as other neoantigens. Likewise, FIG. 11 shows RB1, a classic tumor suppressor but its mutations are inactivating (truncating) and thus, this is not highly useful as a neoantigen. FIG. 11 shows the neoantigen TP53. TP53 is mutated in many forms of cancer. Multiple hotspots such as R282W and R175H, R248L/W and 3 others make this a useful neoantigen.

Next, we select mutation hotspots within the above genes. Not all hotspots are reported below as some contain stop codons. We have selected 8 neoantigens hotspots with a total of 17 amino acid changes: BRAF: V600E; EGFR: A289I A289N A289T A289V; IDH1: R132G R132H; NF1: L844F L844P; PDGFRA: E229K; PIK3CA: E545A E545K; PIK3R1: G376R; TP53: R175H R248L R248W R282W. The selected neoantigens and mutational hotspots cover 58 of 291 (20%) Glioblastoma patients in the cohort and at least one binds the patient's MHC but will not generate T cells cross-reacting with wild-type protein. Some patients have more than one mutation (e.g. one patient has both IDH1 and EGFR mutations). We could also add in the recombination peptide EGFR/TAC-3 (NKB) to this panel of point mutations. See FIG. 12.

We generate 25-30 mer peptides covering these mutations and a corresponding set of peptides covering wild normal sequence: that is, two-17 peptide mixtures, one representing the mutations and the other representing the concomitant wild-type sequences. Then we use these peptides to expand T cells obtained from PBMCs in the blood of Glioblastoma patients. The ICS interferon gamma/TNF or CD107a or killing assay indicates successfully expanded T cells specific for these neoantigen mutations. As discussed above, other than IDH1, other mutations are not correlated with survival.

Preferred neoantigens demonstrate time course stability for the selected alterations, i.e. founder versus late event, and association with expression. We now examine if the hotspots selected in glioblastomas overlap with hotspots in other gliomas and other tumor types.

A cocktail of at least 1 peptide for each of the above mutations covers 96% of Low Grade Gliomas (mostly due to IDH1). A more extensive list, using more comprehensive lists of driver genes using panglioma data can be developed.

In other tumors: the same combination of neoantigens with their associated mutational hotspots cover:100% hairy cell leukemias, due to BRAF; 40% melanomas, due to BRAF; and 7% Lung Squamous Cell Carcinomas due to several hotspots.

Using our methods it is possible to generate a pan-cancer cocktail of neoantigens. These reflect recurrent point mutations occurring at hot spots, and fusion proteins occurring at recombination hotspots that are early events/founder events in tumor evolution (shared between all branches, primary, recurrence, metastasis) and are not clonal but, rather, present in all cancer cells in the tumor and are highly expressed.

The most common mutational hotspots found in human cancer across different tumors were searched in public databases. 41 cancer types were analyzed for the presence of the 100 most commonly mutated hotspots (see FIG. 13). These hotspots are used to create a new panel of neoantigens which are shared across patients and phylogenetically conserved between primary, recurrence and metastases or circulating tumor DNA in the blood. T Direct expansion is performed with this Panel of pre-synthesized neoantigen peptides to expand neoantigen specific T cells (4 weeks). This is an analysis of the most common mutational hotspots found in human cancer. These mutational hotspots are found across different tumors. In particular, an article in Nature Biotechnology 34, 155-163 (2016) (incorporated herein by reference) identifies mutational hotspots in 11,119 human tumors, spanning 41 cancer types, and identified 470 somatic substitution hotspots in 275 genes.

1. A method for making a composition comprising T-cells, the method comprising the steps of:
(a) obtaining an initial cell population comprising T-cells;
(b) stimulating the T-cells by exposing the cell population to one or more target antigens and to cytokines,
(c) culturing the cell population in media comprising cytokines;
(d) testing the cell population for antigen-specific reactivity; and
(e) harvesting the resulting composition comprising T cells.

2-3. (canceled)

4. The method of claim 1, wherein the cytokines in steps (b) and (c) individually comprise one or more of IL-2, IL-7, IL-15, and IL-21.

5. The method of claim 1, wherein the cytokines in steps (b) and (c) individually comprise IL-7 and IL-15.

6. (canceled)

7. The method according to claim 1, wherein the method further comprises polyclonal stimulation of the T cells in the cell population.

8. The method according to claim 7, wherein the polyclonal stimulation comprises exposing the cell population to tetrameric antibodies that bind CD3, CD28 and CD2 after step (c).

9. The method according to claim 1, wherein the cell population is divided into multiple sub-populations, which are each stimulated by exposure to different target antigens.

10. The method of claim 9, wherein the multiple sub-populations are combined prior to step (c).

11. The method of claim 9, wherein the multiple sub-populations are combined prior to step (e).

12. The method according to claim 1, wherein the one or more target antigens comprises a plurality of overlapping peptides derived from the one or more target antigens.

13. The method of claim 12, wherein the one or more target antigens comprise polypeptides derived from a group consisting of one or more sub-dominant antigens, one or more neoantigens, or one or more viral antigens.

14-18. (canceled)

19. The method according to claim 1, wherein the T cell composition resulting from the method comprises greater than 70% CD3+ T cells with predominantly CD8+ versus CD4+ T cells.

20. The method according to claim 1, wherein the T cell composition resulting from the method wherein greater than about 1% of the total CD3+ cells have reactivity toward the antigen or antigens.

21. The method according to claim 1, wherein the T cell composition resulting from the method comprises T cells having elevated surface expression of CD62L, CCR7 or CXCR3 and decreased surface expression of one or more activation/exhaustion markers LAG3, CD244(2B4), CD160, TIM-3, CTLA-4.

22. A method for making a composition comprising T cells, the method comprising the steps of:
(a) obtaining an initial cell population comprising T-cells;
(b) selecting T cells based on expression of T cell activation markers,
(c) performing polyclonal stimulation of T cells, and
(d) harvesting the resulting composition comprising T cells.

23. The method of claim 22, wherein the method further comprises stimulating the T-cells by exposing the cell population to one or more target antigens and to cytokines prior to step (b).

24. The method according to claim 23, wherein step (b) is performed on the initial cell population.

25. The method of according to claim 23, wherein step (b) is performed about 7 days after stimulating the T-cells by exposing the cell population to one or more target antigens and to cytokines.

26. The method of 23, wherein the cytokines comprise one or more of IL-2, IL-7, IL-15, and IL-21.

27. The method of 23, wherein the cytokines comprise IL-7 and IL-15.

28. The method according to claim 22, wherein the T cell activation markers in step (b) comprises one or more of CD69, CD279(PD-1), CD223(LAG3), CD134(OX40), CD183(CXCR3), CD27(IL-7Rα), CD137(4-1BB), CD366(TIM3), CD25(IL-2Rα), CD80, CD152(CTLA-4), CD28, CD278(IOS), CD154(CD40L), and CD45RO).

29. The method according to claim 22, wherein the polyclonal stimulation comprises exposing the cell population to tetrameric antibodies that bind CD3, CD28 and CD2 after step (b).

30. The method of claim 23, wherein the one or more target antigens comprises a plurality of overlapping peptides derived from the one or more target antigens.

31. The method of claim 30, wherein the one or more target antigens comprise polypeptides derived from the group consisting of one or more sub-dominant antigens, one or more neoantigens, or one or more viral antigens.

32-36. (canceled)

37. The method according to claim 1, wherein the T cell composition resulting from the method comprises greater than 70% CD3+ T cells with predominantly CD8+ versus CD4+ T cells.

38. A method for immunotherapy comprising administering to a patient in need thereof a composition comprising T cells wherein the composition is made by the method according to claim 1.

39-42. (canceled)

43. A method for immunotherapy comprising administering to a patient in need thereof a composition comprising T cells wherein the composition is made by the method according to claim 22.
